(19) United States
(12) Patent Application Publication
(10) Pub. No.: US 2016/0199029 A1
(43) Pub. Date: Jul. 14, 2016

(54) ULTRASOUND APPARATUS, SYSTEM, AND METHOD

(71) Applicants: UNIVERSITY OF UTAH RESEARCH FOUNDATION, Salt Lake City, UT (US); STICHTING KATHOLIEKE UNIVERSITEIT (D/B/A RADBOUD UNIVERSITY MEDICAL CENTRE), Nijmegen (NL)

(72) Inventors: Pieter C. STRUIJK, Nijmegen (NL); Edward B. CLARK, Salt Lake City, UT (US)

(21) Appl. No.: 14/913,280

(22) PCT Filed: Aug. 19, 2014
(86) PCT No.: PCT/US14/51636

§ 371 Date: Feb. 19, 2016

Related U.S. Application Data
(60) Provisional application No. 61/867,214, filed on Aug. 19, 2013.

(51) Int. Cl. A61B 8/00; A61B 8/08; A61B 8/04; A61B 8/02; A61B 8/14; A61B 8/06

(52) U.S. Cl. CPC A61B 8/4477; A61B 8/14; A61B 8/06; A61B 8/461; A61B 8/04; A61B 8/02; A61B 8/0866; A61B 8/0891; A61B 8/4488; A61B 8/4494

(57) ABSTRACT

An ultrasound transducer system. The system includes at least three ultrasound transducer arrays including a central transducer array and at least two lateral transducer arrays located adjacent the central transducer array, wherein the at least three ultrasound transducer arrays are arranged such that ultrasound beam paths of the at least three ultrasound transducer arrays overlap in an approximately planar location.

### CROSS-REFERENCE TO RELATED APPLICATIONS

This application claims priority to U.S. Provisional Patent Application No. 61/867,214 filed Aug. 19, 2013, the content of which is incorporated herein by reference in its entirety.

### INTRODUCTION

The present invention relates to ultrasound devices and methods.

At 11 weeks of gestation the crown-rump length of the human fetus is approximately 4 cm and the weight is 7 grams, increasing to 16 cm and 300 grams at mid gestation (20 weeks). At birth the average new born weight is 3500 grams. To assess fetal anatomy and function throughout pregnancy, and bearing in mind the inaccessibility and the vulnerability of the human fetus, the physical principles that can be safely applied are mainly limited to ultrasound applications. However, the anisotropic nature of ultrasound, the angle dependency of Doppler velocity imaging, and the limited spatial and temporal resolution available on current devices contribute significantly to the causes of many prenatally undetected congenital defects. To take one example out of many, between 30% and 60% of congenital heart defects are undetected until after birth, notwithstanding the fact that congenital heart defects are the leading cause of all infant death in the United States. The prenatal diagnoses of congenital abnormalities or fetal disease can make the difference between intra-uterine or infant death and full lifetime expectancy. The current state of the art of medical equipment applicable for fetal diagnosis does not give enough information to assess the well-being of the fetus and thus there is an urgent need to collect more medical information from the distressed fetus to make an informed decision on treatment or timing of birth.

### SUMMARY

In one embodiment, an ultrasound transducer system. The system includes at least three ultrasound transducer arrays including a central transducer array and at least two lateral transducer arrays located adjacent the central transducer array, wherein the at least three ultrasound transducer arrays are arranged such that ultrasound beam paths of the at least three ultrasound transducer arrays overlap in an approximately planar location.

In another embodiment an ultrasound transducer. The ultrasound transducer includes at least three ultrasound transducer arrays including a central transducer array and at least two lateral transducer arrays located adjacent the central transducer array, wherein the at least three ultrasound transducer arrays are arranged such that ultrasound beam paths of the at least three ultrasound transducer arrays overlap in an approximately planar location.

In yet another embodiment, a method of measuring fetal blood pressure. The method includes the steps of: providing an ultrasound transducer having at least three ultrasound transducer arrays including a central transducer array and at least two lateral transducer arrays located adjacent the central transducer array, wherein the at least three ultrasound transducer arrays are arranged such that ultrasound beam paths of the at least three ultrasound transducer arrays overlap in an approximately planar location; obtaining a two-dimensional image of a fetal aorta lumen using the ultrasound transducer; displaying the two-dimensional image to a user; obtaining from a user a location of a center of the fetal aorta lumen; generating a center array echo line from the central transducer array and a plurality of side array echo lines from each of the lateral transducer arrays, wherein each of the center array echo lines and the side array echo lines cross at the location; obtaining ultrasound data from each of the center array echo line and the plurality of side array echo lines; and determining fetal blood pressure using the ultrasound data.

In still another embodiment, a method of determining a thickness of a fetal aorta wall. The method includes the steps of: obtaining a plurality of ultrasound scans through the fetal aorta wall, wherein each of the plurality of ultrasound scans has a near wall reflection point and a far wall reflection point; aligning each of the plurality of ultrasound scans according to the near wall reflection point in each of the plurality of ultrasound scans to produce a near wall alignment; determining a near wall reflection mean from the near wall alignment; decomposing the near wall reflection mean into a near wall inner Gaussian pulse and a near wall outer Gaussian pulse; and determining a thickness of the near wall based on the near wall inner Gaussian pulse and the near wall outer Gaussian pulse.

In a further embodiment, a method of displaying multi-angle ultrasound data from a fetus. The method includes the steps of: providing an ultrasound transducer having at least three ultrasound transducer arrays including a central transducer array and at least two lateral transducer arrays located adjacent the central transducer array, wherein the at least three ultrasound transducer arrays are arranged such that ultrasound beam paths of the at least three ultrasound transducer arrays penetrate the fetal tissue and structures from different angles and overlap in an approximately planar location; obtaining two-dimensional images of the tissue using at least two of the ultrasound transducer arrays; and combining the two-dimensional images to provide a composite image of the tissue and structures.

Other aspects of the invention will become apparent by consideration of the detailed description and accompanying drawings.

### BRIEF DESCRIPTION OF THE DRAWINGS

FIG. 1a shows views of an ultrasound device in accordance with embodiments of the invention.

FIG. 1b shows ultrasound beam paths in a triple-scanning mode.

FIG. 1c shows various ultrasound beam paths in single- and double-scanning modes.

FIG. 1d shows several ultrasound transducer arrangements.

FIG. 1e shows an ultrasound device in accordance with embodiments of the invention.

FIG. 2 shows a diagram of beam paths at a single point within a fetal aorta sample using an ultrasound device in accordance with embodiments of the invention.

FIG. 3 shows a screen layout in accordance with embodiments of the invention, where the top portion of the image shows a two-dimensional image from a single transducer and the lower portion shows M-mode images from the region outlined in the two-dimensional image.

FIG. 4 shows presentation of the fetal aortic flow-area loop.

FIG. 5 shows a four element fetal aortic downstream impedance model as an equivalent circuit (left) and a graph showing that the model fits data obtained from a human fetus (right).

FIG. 6 shows an intensity-averaged image from all 164 frames collected in a 2-s acquisition period from a human fetus.

FIGS. 7a-7h show tracked aortic wall positions for data collected from a human fetus.

FIG. 8 shows positions of the intima (media)-blood interface frame by frame along the scan lines that exhibit maximum wall reflections for the longitudinal and cross-sectional planes, respectively (top) as well as the distances between the interfaces (bottom).

FIG. 9 shows a Bland-Altman plot of fetal aortic diameters derived from the longitudinal and cross-sectional planes, where the dotted lines represent the 95% limits of agreement, estimated as the mean difference ±1.96 X standard deviation of the differences. No significant bias is present as indicated by the solid line close to zero, representing the mean difference.

FIG. 10 shows Bland-Altman plots of fetal aortic pulse wave velocity (PWV) assessment between observations. The left and middle panels (observers 1 and 2, respectively) indicate agreement within observers, and the right panel, agreement between observers. The top and bottom lines indicate the 95% limits of agreement, estimated as the mean difference ±1.96 X standard deviation of the differences. No significant bias is present as indicated by the mean lines in the middle that almost coincide with the zero line.

FIG. 11 shows pulse wave velocity (left), end-diastolic fetal aortic lumen diameter (center), and pulse diameter (right) data with superimposed 10th, 50th and 90th percentile lines; as the absolute residuals from linear regression analysis indicated no relation with gestational age, all percentile lines could be linearly described.

FIG. 12 shows the calculated distensibility coefficient (left), local fetal aortic compliance (center), and pulse pressure (right) with superimposed 10th, 50th and 90th percentile lines.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

In various embodiments, the methods and systems disclosed herein may be implemented on one or more computer systems. Each computer system may be in wired or wireless communication with one another through a combination of local and global networks including the Internet. Each computer system may include one or more input device, output device, storage medium, and processor/microprocessor. Possible input devices include a keyboard, a computer mouse, a touch pad, a touch screen, a digital tablet, a microphone, a track ball, and the like. Output devices include a cathode-ray tube (CRT) computer monitor, a liquid-crystal display (LCD) or LED computer monitor, touch screen, speaker, and the like. Storage media include various types of local or remote memory devices such as a hard disk, RAM, flash memory, and other magnetic, optical, physical, or electronic memory devices. The processor may be any typical computer processor for performing calculations and directing other functions for performing input, output, calculation, and display of data in accordance with the disclosed methods. In various embodiments, implementation of the disclosed methods and systems includes generating sets of instructions and data (e.g. including image data and numerical data) that are stored on one or more of the storage media and operated on by a controller.

In some embodiments, implementation of the disclosed methods may include generating one or more web pages for facilitating input, output, control, analysis, and other functions. In other embodiments, the methods may be implemented as a locally-controlled program on a local computer system which may or may not be accessible to other computer systems. In still other embodiments, implementation of the methods may include generating and/or operating modules which provide access to portable devices such as laptops, tablet computers, digitizers, digital tablets, smart phones, and other devices.

In western society, the age at which women become pregnant is increasing. Consequently, the rate of complications is also increasing. Monitoring of the unborn child is more common than a decade ago and echographic examination at 20 weeks gestation is a standard procedure and in some countries a monthly echogram is usual. During pregnancy, hypertension of the fetus and/or mother may develop and is associated with fetuses that are small for their gestational age and/or premature neonates. Since fetal hypertension may precede the development of maternal hypertension, it is important to diagnose and treat fetal hypertension as early in pregnancy as possible. However, non-invasive arterial pressure measurement in the fetus is not possible with current technology. On the other hand, the device disclosed herein accurately estimates fetal blood pressure and fills a social need in developed as well as underdeveloped countries all over the world.

In addition, the health care cost economic benefits are enormous. The costs for one month at a neonatal intensive care are in the order of magnitude of $45,000 in the USA as well as in most part of Europe. Furthermore, the POPS study (project on preterm and small for gestational age infants) has shown that these children have an increased risk for hypertension, cardiovascular disease, diabetes, and obesity. Additionally, the cognitive development of these children is also severely affected as demonstrated by the fact that 27 percent of this population needs special education. Timely diagnosis of fetal hypo- or hypertension coupled with effective therapy will likely improve fetal outcome and reduce medical costs.

Non-invasive pressure measurement is a technique which might be used for a number of other applications in newborns, children and adults as well. Among many other examples it is likely useful in monitoring pulmonary hypertension, a growing problem in Western Society that may lead to right-sided heart failure if not properly treated.

Although human fetus pulse pressure has been estimated, it has not been possible to determine or estimate the mean pressure accurately. Moreover, known methods cannot be used in the first half of pregnancy, as sufficient aortic length is lacking early in pregnancy when the fetus is smaller. On the other hand, using the presently-disclosed apparatus and methods, fetal pressure estimation can be determined based on pulse wave velocity, blood flow and diameter data obtained from the fetal aorta and by applying the so-called Windkessel model.

Accordingly, disclosed herein is a unique high-sensitivity triplet ultrasound transducer with dedicated beam steering and sequencing software which satisfies the necessary specifications concerning spatial and temporal resolution as well as phase stability to achieve anisotropic compensated brightness mode imaging, angle-independent imaging of blood and tissue velocity, and tissue strain analysis. This system has been used to determine a number of fetal hemodynamic parameters including fetal aortic blood pressure.

Disclosed are apparatus, systems, and methods for determining hemodynamic parameters, including blood pressure, in particular from a fetus in utero. In some embodiments, the apparatus may include a unique ultrasound transducer having a curved array transducer and a pair of phased-array transducers positioned adjacent to and on opposite sides of the curved array transducer is used to obtain raw data. The curved array transducer in such embodiments is used to determine an initial location of the fetal aorta. The position of the transducer is adjusted such that one beam of the curved array transducer is approximately perpendicular to the wall of the aorta. A user identifies the location of the center of the aorta along this beam, using an interactive graphical user interface. The beams of the phased array transducers are steered so that they intersect with the curved array beam at the center of the aorta. The system obtains raw data (e.g. several seconds) from the ultrasound transducers, which is then used to determine hemodynamic parameters. Among the parameters that are obtained from the data are the wall thickness and lumen inner diameter, which are obtained from the decomposition of wall reflections into separate Gaussian pulses representing the thickness of the walls.

In various embodiments, a functional model of the aortic downstream impedance can be applied using the present system in order to approximate peripheral resistance and arterial compliance. The aortic volume flow and cross-sectional area waveforms may be used as input to the model, resulting in the magnitude and shape of arterial blood pressure as output of the model.

Disclosed herein are apparatus and methods for functional imaging and for measuring hemodynamic parameters including blood pressure in the human fetus.

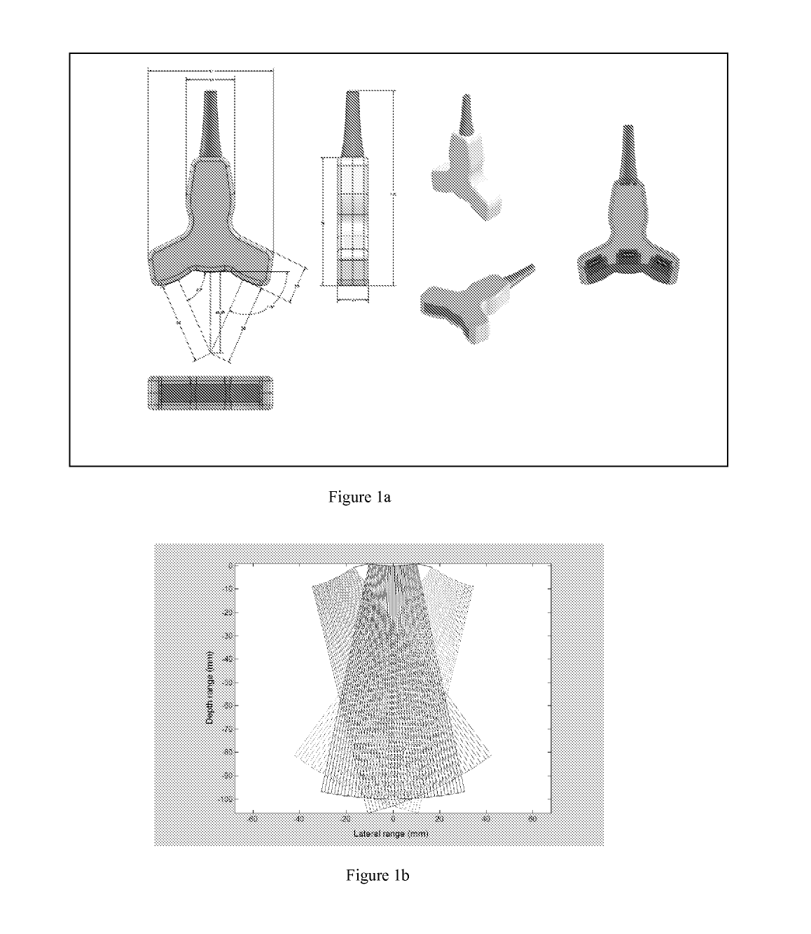

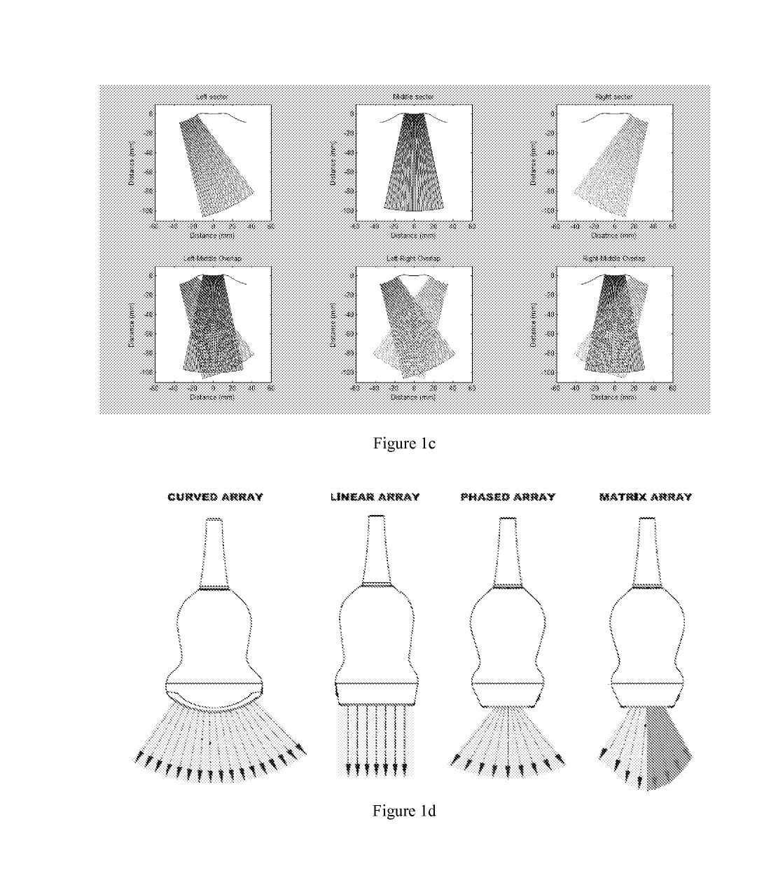

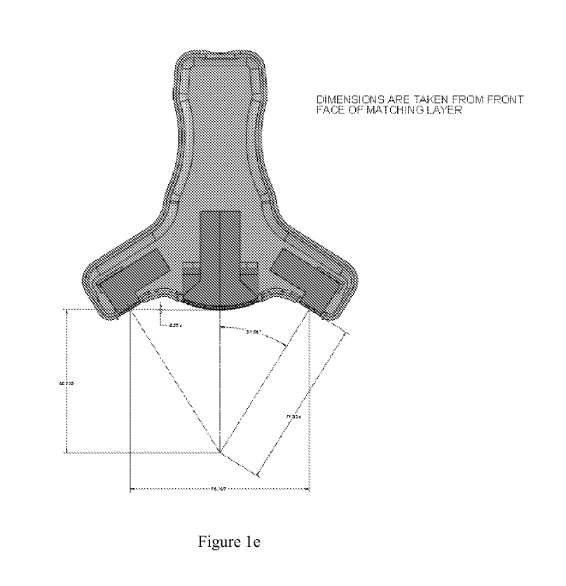

An embodiment of the presently-disclosed triplet ultrasound transducer is illustrated in FIGS. 1a, 1b, 1c, 1e. In one particular embodiment, the transducer includes three convex curvilinear arrays of elements for imaging anatomical structures from three different viewing angles. Due to the anisotropic nature of ultrasound the three conventional brightness mode (B-mode) images will be different from one another; in some embodiments, the B-mode images from the different viewing angles can be combined to produce a composite image. An ultrasound echo from a fiber-rich structure will be strongest if the incident angle is perpendicular to the structure but weaker at any other incident angle. In contrast, small particle-rich structures such as blood are less angle dependent. Instead of conventional B-mode imaging, angle-corrected imaging can be achieved from the overlapping sector scans. Instead of imaging based solely on angle-dependent echo amplitude, embodiments of the present invention present anatomical structure-related fetal imaging based on both echo amplitude and angle dependency as well as additional functional parameters.

Using the proposed triplet transducer at mid-gestation acquisition, frame rates between 80 Hz and 200 Hz are feasible, although rates of up to 300 Hz may also be possible. Frame rates in this range are adequate to calculate the tissue velocity vector on every cross point of two echo lines in the viewing plane. This implies that deformation parameters such as tissue velocity, strain, and strain rate can be added to the parameter list for structural and functional related image codes. Besides morphological information, deformation parameters provide important information regarding quantization of myocardial function.

By increasing the number of ultrasound pulse emissions, angle-independent blood flow velocity and tissue velocity can be measured simultaneously on every cross point of two echo lines. Blood flow velocity in the heart, arteries, and veins are characterized by low echo amplitude and high velocities compared with surrounding tissue such as myocardium and arterial and venous walls, which exhibit high echo amplitude and low velocities. Embodiments of the triplet transducer design allow development of an imaging code based on echo amplitude from three directions, angle dependency, blood velocity, tissue velocity and strain analysis. Angle-independent blood flow velocity measurements provide important information concerning organ perfusion and cardiac function. Moreover, the triplet device is equipped with a feature such that, by marking a single spot, the number of color Doppler lines will be automatically reduced to two lines which cross at the region of interest and the velocity vector and velocity waveform from the region of interest will be presented at sufficient temporal resolution to allow clinical diagnostics.

Hemodynamic Parameters and Fetal Pressure Measurements

In various embodiments the disclosed system includes an intelligent graphical user interface that guides the operator as he or she images the fetal aorta. Once the center of the aorta is identified by a user and marked by a mouse click, flow velocity is presented in color showing time-based motion mode (M-mode) information simultaneous with near and far aortic wall movements, enabling the operator to optimize the probe position relative to the fetal aorta, in particular enabling the user/operator to position the probe so that the center is approximately perpendicular to the aorta. Subsequently, a command may be issued to start a raw data acquisition period (e.g. two seconds). In embodiments of the acquisition mode, three ultrasound beams may be generated which cross at the center of the fetal aorta. Directly after data acquisition, the aortic flow and blood pressure waveforms are displayed along with a listing of one or more of the following hemodynamic parameters: 1) fetal heart rate, 2) aortic wall thickness, 3) time averaged aortic lumen diameter, 4) pulse wave velocity, 5) local aortic distensibility coefficient, 5) fetal aortic compliance coefficient, 6) elastic modulus of the fetal aortic wall, 7) mean aortic blood flow, 8) stroke volume, 9) downstream peripheral resistance, 10) compliance of the fetal vascular bed, as well as 11) systolic, 12) diastolic, and 13) mean fetal aortic blood pressure.

During prenatal development, form and function are strongly related to each other. For instance the early human heart is functioning before structural cardiac morphogenesis is complete. Evidence exists that cardiac function itself strongly regulates the transformation from a single muscle-wrapped tube into a four-chamber heart. Meanwhile, diminished cardiac function will influence prenatal development of other organs or vice versa.

The disclosed ultrasound prenatal diagnostic device simultaneously enables functional and morphological investigation to more carefully study prenatal cardiac structure and function. Factors that have previously inhibited development of an ultrasound device that allows both functional and morphological fetal examination include the fact that:
Traditional imaging is confounded by the anisotropic nature of ultrasound. The device disclosed herein images an anatomical structure from three different angles. The applied signal analysis techniques appropriately compensate for the anisotropic effect of ultrasound.
Single sector imaging can generate unacceptable images due to shadowing from the ribs. As in adults, at later gestational ages the fetal ribs will reflect most of the acoustic energy and a large shadow may be presented behind the ribs using single sector scanning. The triple sector scanning will provide information from three different angles, and further information may be available from ultrasound bursts that travel between ribs. The multi-angle approach may help to diminish the shadowed zone.
Single sector scanning cannot generate angle-corrected color Doppler (or otherwise-coded) images. Traditional color Doppler imaging is confusing as the velocity coding is based on the velocity component in the direction of the ultrasound beam. Consequently, velocities are presented between zero and plus or minus their actual magnitude and therefore contains no medical information as long as the Doppler angle is unknown. The presently-disclosed invention allows true velocity magnitude color coding.
Traditional fetal imaging is not enhanced by functional parameters. By applying cross correlation-based computational methods on the Radio Frequency (RF) data, fetal blood velocity and tissue displacement at the sub-micrometer level is available in the 2D image or cineloop from the triplet ultrasound device. The local functional parameters such as blood flow velocity or strain information will be presented numerically by a single mouse click or, in the case of a cineloop, as a waveform.
Angle-dependent imaging precludes fetal structure-related imaging. The invention disclosed herein offers the opportunity to develop imaging codes that are fetal structure- and/or fetal function-related.

To date, factors that have made it difficult to develop a device to measure fetal blood pressure include:
Fetal arterial pressure cannot be measured directly because of unacceptable risk to the fetus. This invention describes a new indirect method that estimates fetal blood pressure from simultaneously-derived fetal aortic blood flow and cross-sectional area waveforms.
A device to simultaneously measure pulse wave velocity, blood flow and vessel diameter has not previously existed.
As opposed to the Doppler angle for blood flow velocity measurements, which should be 70 degrees or less, the angle of incidence of the ultrasound beam and fetal aortic wall should be perpendicular (i.e. at an angle of approximately 90 degrees to the fetal aortic wall) to assure accurate diameter determination. Moreover, it is of utmost importance that the phase relationship between the flow and area waveforms be preserved. The presently-disclosed invention describes a new dedicated transducer design to assure these preconditions.
The depth of the fetus within the maternal abdomen may vary by a considerable amount. During advanced pregnancy, and in some cases in combination with maternal obesity, the fetal aorta might be presented at a depth of 15 cm or more, while the distance between the transducer face and the fetal aorta might be 4 cm or less in a thin mother early in pregnancy. This wide range of depths requires automatic optimization of a large number of parameters such as ultrasound pulse length, frequency, focusing, beam steering, time gain control etc. Various embodiments of this invention include dedicated software designed to fulfill this task.
Estimates of blood flow velocity require complex evaluation. A number of blood flow velocity estimators are described in the literature. In some embodiments, the complex cross-correlation model is used to estimate velocity, as this model uses minimal model assumptions. In addition, this model provides the center frequency and the bandwidth of the emitted ultrasound pulse at the region of interest, information which is used to accurately measure the fetal aortic diameter.
Prior techniques did not define the surface of the aortic intima. Another aspect of the invention is a new method to determine the intima media (wall) thickness of the fetal aorta by decomposing the aortic wall reflections into two Gaussian pulses. As this method accurately determines the blood intima interface, the inner diameter of the aorta can be measured more accurately than has previously been possible.
Pulse wave velocities measured by slow repetition rates are confounded by impedance mismatch. Due to the elasticity of the arterial vascular bed, the pressure wave propagates through the arterial system and will be reflected in part by any impedance mismatch such as bifurcations. For this reason, only the early onset of the systolic phase of the blood flow and cross-sectional area waveforms are undisturbed by wave reflections. This implies that high acoustic burst repetition rates are needed to achieve high temporal resolution. Thus, it is particularly important to estimate the key parameter to estimate fetal blood pressure, namely, the pulse wave velocity. The unique combination of sequencing software and transducer design provides appropriate pulse wave velocity estimations.
Ultrasound safety requires high ultrasound transducer sensitivity. To stay well within the acoustic safety limits at high burst repetition frequencies, the triplet ultrasound transducer is designed by applying high-sensitivity ultrasound technology. Moreover, to meet the ALARA (As Low As Reasonably Achievable) principle, the duration of the high burst rate is limited to 2 seconds.
The fetal aorta is curved. The blood flow direction cannot be assumed to be straight, either at the level of the aortic arch or at any level of the descending aorta, the latter mainly as a consequence of the typical fetus position. The applied cross beam method allows measurement of blood velocity as well as velocity direction (velocity vector). Thus, the combination of the pulsatile nature of aortic flow and the curved and tapered shape of the fetal aorta confuses the velocity profiles. The presently-disclosed invention ensures accurate mean velocity estimation by acquiring the velocity profiles at high temporal resolution as well as at high spatial resolution across the lumen of the fetal aorta.
The fetus is in motion. The fetus can move freely and its behavior cannot be influenced. Therefore, to avoid fetal movement artifacts, a user-friendly and intelligent graphical interface is needed to provide the ultra-sonographer with real-time, relevant information to be able to quickly optimize the probe position when the fetus is at rest.

FIG. 1a shows several different views of a multi-directional transducer according to embodiments of the invention. The multi-directional transducer of FIG. 1a includes three curvilinear arrays of transducer elements arranged in a single plane such that the scanning regions of the curvilinear arrays overlap in an approximately planar region adjacent to the transducer (e.g. at distances ranging from 1-30 cm from the transducer). As shown in FIG. 1a, the two side arrays are generally symmetrically angled relative to the center array, although in various embodiments the outer dimensions can vary depending on the application. In certain embodiments, a centerline of each of the transducer arrays crosses at a point that is between 3-25 cm from the transducers and the centerlines are approximately 20°-30° (in some embodiments 25°) apart as measured from the point of intersection, i.e. the centerlines of the two lateral transducer arrays in a triplet embodiment are 20°-30° apart from the centerline of the central transducer.

While a number of the illustrated embodiments depict a triplet transducer, i.e. a transducer having three separate transducer arrays, in various other embodiments the transducer may have other numbers of transducer arrays, including two, four, five, or more, which produce two, four, five, or more separate scans such as those shown in FIG. 1b. In yet other embodiments, a single transducer array may be used to generate ultrasound data that is comparable to that obtained using the multi-transducer (e.g. triplet transducer) arrangement. For example, a single transducer element may contain a series of independently-controllable elements that are formed into an arrangement that functions in a similar manner to the triplet transducer shown in FIG. 1a. That is, the single transducer may have three curvilinear sections that are angled relative to one another as shown in FIG. 1a and the individual elements of the single transducer may be independently controlled so as to produce separate scans such as those shown in FIG. 1b. In other embodiments the two, three, or more elements may be generated by two or more separate transducers. In each such embodiment, the separate transducer arrays (or alternatives to separate transducer arrays that are produced for example by separately controlling groups of elements within a single transducer but which generate similar data) are arranged such that the scanning regions of the curvilinear arrays overlap at a distance from the transducer.

In various embodiments, each of the transducer arrays (or alternatives thereto, e.g. in a single-element embodiment) is curvilinear. In some embodiments, the central transducer array is curvilinear while one or more lateral arrays are straight. In other embodiments, one or more of the curvilinear arrays within a given transducer has a different curvature than the others. The curvilinear transducer arrays produce a fan-shaped scan (e.g. as shown in FIGS. 1b, 1c) and may have a radius of curvature ranging from 20-70 mm (50 mm in certain embodiments).

In certain embodiments, the transducer arrays are set into a housing (e.g. made of a medical grade plastic or other material) which holds the transducer array elements in place. In one embodiment the housing is approximately 10 cm wide (i.e. side to side in the top left panel of FIG. 1a)×2.4 cm thick and the individual transducer arrays are approximately 2 cm wide with sector angles of approximately 25°. The housing includes suitable electronics to couple the transducers to a controller, where the controller in turn controls the ultrasound transducers and collects echo data. The collected data is processed and/or transmitted by the controller, e.g. to display images and other information to a user. The controller also collects input from users such as a location of the center of the aortic lumen.

FIG. 1d shows various ultrasound transducer arrangements, which in some embodiments can be utilized as part of a multi-directional transducer.

The curved/curvilinear array (FIG. 1d) of transducer elements performs the sector scanning of ultrasonic beams without exciting the transducer elements with different timing relations. The delayed timing is the technique applied in phased array transducers and responsible for generating so-called “grating lobes” (grating lobes are energy peaks or artifacts that may exist outside the center of the beam). In some cases, curvilinear array transducers are not suitable for cross beam applications and the beams can be steered only by very small angles. As disclosed herein, beam steering may be used in some cases for fine tuning the beam.

The linear array transducer (FIG. 1d) may be used for non-invasive blood pressure estimation in superficial blood vessels of newborns, children, and adults. For superficial arteries, high frequency linear transducers can be applied which clearly shows the intima media layer at perpendicular incident angles, while at non perpendicular insonation the layers are not distinguishable from each other.

The phased array transducer (FIG. 1d) is similar to a linear array transducer but having a small footprint. All elements of the array are used to steer a bundle of ultrasonic beams. One or more phased array transducer could be used in place of the disclosed curvilinear transducers.

The matrix array transducer (FIG. 1d) can be used for volume scanning. In some embodiments, matrix transducers may be used in place of the curvilinear array transducers. Since the matrix transducer can function as a 2D phased array transducer, using matrix transducers in some embodiments may operate in a similar manner to using phased array transducers. In addition, matrix transducers can also generate extra lines (or planes) in the elevation direction to detect or compensate for off-plane movements, although this may lead to a loss of scan/repetition rate that is proportionally reduced by the number of scan lines used in the off-plane direction.

FIG. 1e shows an embodiment of a triplet ultrasound transducer which includes a combination of phased array transducers (left and right) and a curvilinear transducer (center). This embodiment takes advantage of the wide beam steering capacity of phased array transducers (+/−45°) to provide a wider beam coverage area. A relative low center frequency was chosen (2.5 MHz) in this embodiment in order to have sufficient penetration depth even for obese pregnant women.

In various embodiments, the disclosed triplet transducer may be optimized for performance during mid-gestation (second trimester). Modifications to the design may be made to accommodate situations in which the fetus is more difficult to image, for example during first trimester and/or in the case of maternal obesity. For example, higher ultrasonic frequencies such as 7 MHz center frequency (50% bandwidth) may be applied in a first trimester transducer to achieve better spatial resolution, while lower ultrasonic frequencies such as 2.5 MHz center frequency (50% bandwidth) may be applied for a third trimester triplet transducer to achieve more penetration depth at the cost of some spatial and temporal resolution. Based on physical principles the conversion from electrical energy to acoustic energy is band pass filtered. A fractional bandwidth of 50% means that for a 7 MHz transducer, the −6 dB power reduction is at (7-1.75) MHz and (7+1.75) MHz (Bandwidth 3.5 MHz, which is 50% from 7 MHz. Within the bandwidth operators, one can use different emission frequencies. Therefore, in various embodiments relatively large steps are selected for transducers used in different groups of patients: for first trimester 7 MHz, for second trimester 4.5 MHz, and for third trimester and/or obesity 3.5 MHz. Wide bandwidth transducers (fractional bandwidth 50% or more) are generally used in applications such as this to achieve sufficient spatial resolution.

While it may be possible to use current ultrasound transducers to obtain data from superficial blood vessels in newborns, children, and adults, current ultrasound technology is not suitable for obtaining blood pressure measurements in fetuses, due to the depth of the fetus within the mother as well as the small dimensions of the blood vessels within the fetus. The presently-disclosed triplet transducer makes this possible by collecting data from several angles relative to the fetal aorta in order to calculate the various hemodynamic parameters disclosed herein. By adjusting parameters including the ultrasonic frequency range and the number of elements on the transducer arrays, the disclosed transducer can be adapted for different gestational ages and maternal body sizes.

Although present disclosure refers to the use of the disclosed apparatus, methods, and systems on a fetuses in a maternal subject, in various embodiments the subject may be a male or female (pregnant or not) and the tissue that is studied may include other blood vessels within the subject's body.

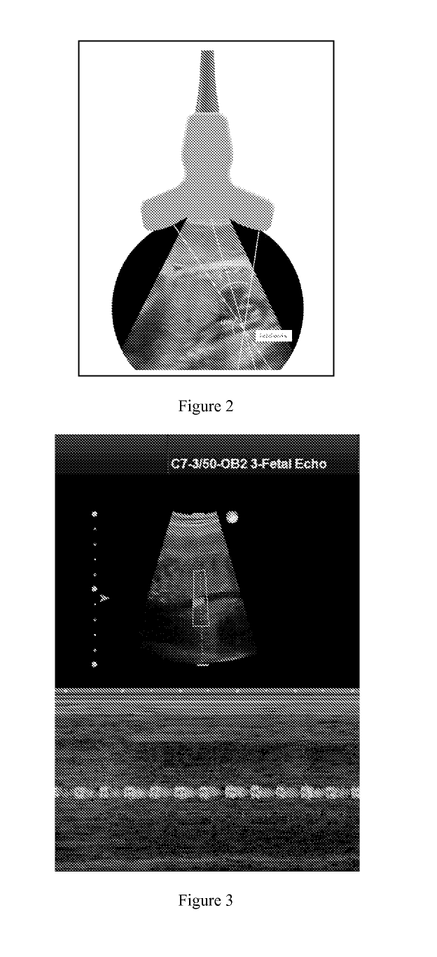

FIG. 1b illustrates a triple scanning mode for a triplet transducer. In various embodiments, the triplet transducer design may be a composite of three separate transducers built into a single housing (FIGS. 1a-1c, 2). The three transducers (arrays) are of the type of convex curved array such as mostly used in obstetrics for fetal scanning. These are wide band transducers, for which the typical center frequency range is between 2 MHz and 7 MHz, particularly for fetal scanning and other obstetrics uses, although in some embodiments the frequency range may be 1-20 MHz, particularly for uses outside fetal scanning and/or obstetrics.

The total number of all elements of the triplet transducer design depends on its application. As soon as the number of elements exceeds convenient cabling, miniaturized electronics technology may be built into the transducer housing to switch between arrays, thereby reducing the number of wires needed to connect the triplet transducer with the ultrasound device. In various embodiments, while increasing the number of elements can lead to improved resolution, this can also slow down the data acquisition rate and so the number of elements should be balanced with the desired speed of acquisition. In some embodiments, a transducer array having many elements may be operated so that not all of the elements are used in order to produce a higher data acquisition rate.

In the example shown the total number of elements is 128. In one embodiment, the number of elements in each of the side arrays is 42 and the center array includes 44 elements. In other embodiments, the total number of elements may be greater than 128, for example 256, 512, or more, particularly when curvilinear transducer arrays are used, where the elements may be distributed among the arrays in different ways. In other embodiments in which matrix array transducers are used, the total number of elements may be several thousand, e.g. if three 32×32 matrix transducers are used then the total number of elements is 3072. The imaging echo lines are shown for the left, middle, and right arrays, respectively.

In the center of the imaging field an area exists in which three lines from the three respective curvilinear arrays cross. Within this area, full anisotropic and shadow compensation as well as structure-related and functional imaging can be realized. A complementary area exists in which at least two lines cross; in this area, anisotropic and shadow compensation is limited by two instead of three vector directions. Areas covered by at least one transducer, whether or not lines cross, are eligible for conventional imaging. A typical examination using a device according to embodiments of the invention may start with a straightforward B-mode averaging algorithm to generate a maximum area image for orientation purposes. Subsequently, the medical personnel can zoom in to a limited region to perform full structure-related and functional imaging.

FIG. 1c illustrates additional imaging modes. The panels in the top row show single sector scanning from three different viewing angles. Maximum frame rates can be achieved by selecting this mode. Two dimensional speckle tracking can be applied in this mode to examine fast moving structures such as cardiac valves.

The panels in the bottom row show the three possible combinations of two overlapping sectors, i.e. scanning patterns which result from activation of two of the three arrays of a triplet transducer. These modes allow for the calculation of the displacement vector at the sub-micrometer level (e.g. 0.1 μm) at each point at which two lines cross. This high spatial resolution is achievable because raw RF ultrasound data is available from two different beam directions. With single sector scanning (as shown in the top row panels), the differences in resolution in the axial and lateral directions greatly impacts the spatial resolution. In various embodiments having different numbers of transducer arrays, other combinations of scanning patterns which use fewer than all of the arrays are also possible (e.g. in an embodiment having four arrays, various scanning patterns using three of the arrays at the same time may be implemented).

In non-invasive fetal pressure measurement mode, the triplet transducer design allows two-dimensional imaging simultaneously with two echo lines originating from the built-in side arrays on either side of the center array. At the crossing of the two side array lines, the velocity vector is calculated by solving for the unknown velocity magnitude (|V|) and direction (θ) from the two respective Doppler equations. The solution can be described as:

$$\vert V \vert = \frac{c}{2 {F}_{0} sin δ} \sqrt{{f}_{1}^{2} + {f}_{2}^{2} - 2 {f}_{1} {f}_{2} cos δ}$$

$$θ = {tan}^{- 1} ( \frac{cos δ - \frac{{f}_{2}}{{f}_{1}}}{sin δ} )$$

where f₁ and f₂ are the Doppler frequency shifts, F₀ is the estimated center frequency of the received echo, δ is the included angle formed by the two beams originating from the two side array transducers, and c is the velocity of sound in tissue (set to 1540 m/s).

The echo line from the center array, which insonates the fetal aorta from an approximately perpendicular angle, is used to track the near and far wall.

FIG. 3 shows an example of a screen layout in which the upper panel shows a 2-D image from a single curved array transducer including the presentation of a single line from which color M-mode is recorded. The lower portion of FIG. 3 shows a time sequence of a 1-D region covered by the dashed line in the center of the image in the upper portion of FIG. 3. When the triplet transducer is applied, two M-mode recordings will be presented instead of one. In addition to the vector color M-mode, the M-mode presentation of the aortic walls as obtained from the selected center line of the triplet center array will be presented as well. The data in FIG. 3 was obtained from a human fetus. In various embodiments, data such as that shown in the lower portion of FIG. 3 may be acquired at a frame rate of about 10-30 Hz.

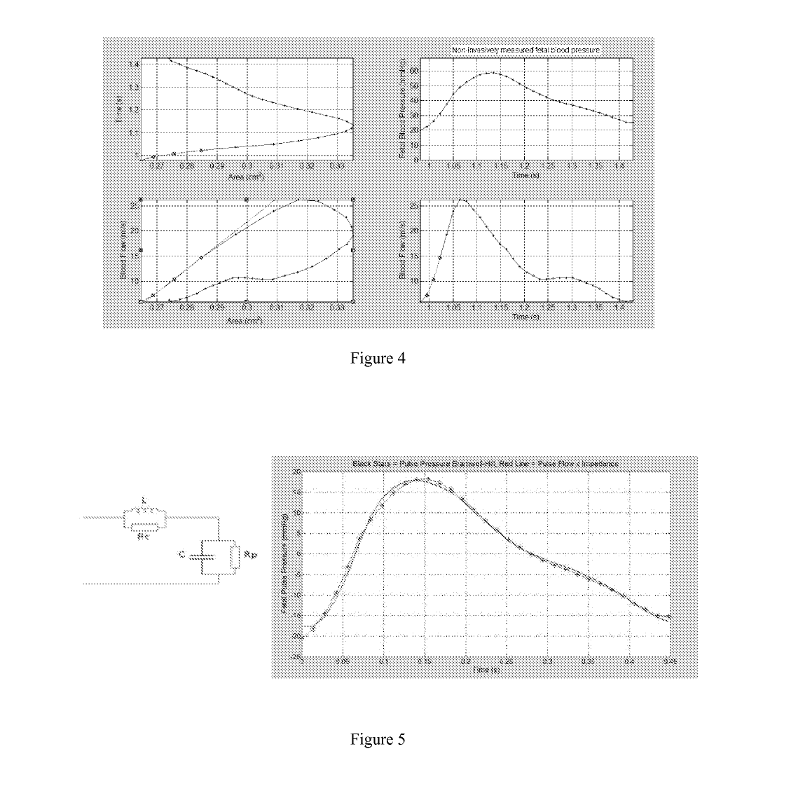

Flow and Area waveforms may be presented together in a single graph using a Flow-Area loop. The examples depicted in FIG. 4 show the flow and area waveform of one complete cardiac cycle. The larger (red) dots and the associated (red) lines represent the linear part of the Flow-Area loop during the early onset of the cardiac cycle, from which the pulse wave velocity can be calculated. The data in FIG. 4 was obtained from a human fetus. The data in FIGS. 3 and 4, which was obtained using a commercially available ultrasound system (Sonix Tablet Research) with the feature of so called “color M-mode imaging” and the ability to collect raw RF data, serves to provide proof of principle. The data demonstrate that one can: simultaneously obtain fetal aortic diameter waveforms and blood flow velocities while preserving their exact time relationship; obtain the Pulse Wave Velocity from a single spot (locally) using an alternative method, the so called Flow-Area loop, which can be applied early in pregnancy; and obtain magnitude and shape of fetal aortic blood pressure by using aortic volume flow and cross sectional area waveforms as input of an appropriate downstream impedance model.

To describe the downstream impedance, a four element lumped-parameter model is applied (FIG. 5). Arterial inertance is added to the three element model that combines the transmission line model with the classical two element Windkessel model. In this model Rₚ represents the peripheral resistance and C the arterial compliance (as in the Windkessel model), R_{c} the characteristic resistance (transmission line element), and L the arterial inertance. Addition of the fourth element (inertance) improves compliance estimation. The graph in the right panel shows an excellent model fit for data obtained from a human fetus.

While the data above help demonstrate the feasibility of determining various fetal hemodynamic parameters, fetal blood pressure itself cannot be accurately derived from a single M-mode line because the inner diameter can only be measured accurately when the insonification angle is perpendicular. However, at a perpendicular insonification angle the blood flow velocity cannot be measured. As a compromise, a Doppler angle close to 90° is typically chosen (e.g. between 70° and 80°) to obtain Doppler velocity information as well as the ability to track the aortic walls. However, the disclosed decomposition method cannot be applied properly using angles between 70° and 80°. As discussed herein, the fetal aorta is curved and therefore it is very difficult to obtain an accurate estimate of the Doppler angle from a 2D image. It should be noted that the scaling of pressure and flow in this data is also limited for the reasons above. Even so, the data provide a good fit for the four-element impedance model, which is scale-independent. Nevertheless, three ultrasound echo lines will ultimately be needed (i.e. one perpendicular to the aorta and two angled lines to obtain velocity vectors) in order to measure fetal blood pressure accurately.

Sequencing for Fetal Pressure Measurement

In various embodiments, a fetal examination may start with two-dimensional scanning in order to locate the fetal aorta, where the two-dimensional scan image is shown on a display. As soon as a clear longitudinal cross section of the aorta is presented, the center of the lumen from which maximum wall reflections are observed is marked via user input, e.g. mouse clicking. Note that maximum wall reflections in the M-mode recording commonly indicate an approximately perpendicular incidence angle. The coordinates of the marked point are used to select the center array echo line exhibiting the marked point and also to calculate the direction of the two side array echo lines in such a manner that the lines cross at this point. The selected point might not coincide with the grid of the image lines, such that fine tuning may be achieved by additional electronic beam steering. The focus point for the two side array lines and the two-dimensional image are automatically set at the depth of the marked point relative to the elements generating the respective lines.

Positioning of the transducer relative to the subject is important for obtaining optimal image and data quality and in various embodiments optimal positioning is achieved by an operator adjusting the position of the transducer using visual feedback with the image on the screen. The two-dimensional blood velocity vector to be calculated from the cross-beam should coincide with the three-dimensional velocity vector in space. This model assumption is valid only if a longitudinal cross-section of the fetal aorta or any other artery is obtained. To be able to verify the model assumption, a real time two-dimensional image showing the longitudinal cross-section should be available (e.g. shown on the display) while setting optimal probe position. FIG. 2 shows an example of such a presentation. Moreover, the aortic wall reflections shown in the M-mode recording should be clearly distinguishable from aortic blood and a good quality vector color Doppler M-mode presentation from the two crossed side array echo lines should be obtained.

After marking the center of the aorta presentation, the 2-D image will be frozen and only the three aforementioned echo lines will be pulsed to achieve maximum temporal resolution for pressure assessment. In various embodiments, data acquisition will stop automatically after 2 seconds; in other embodiments, data may be collected for shorter or longer amounts of time, depending on the number of heart beats needed for adequate analysis, at waveform sampling rates of 10 Hz-300 Hz.

Fetal Aortic Blood Flow Velocity and Aortic Wall Velocity Assessment

The Doppler formula can be applied for fetal aortic blood flow velocity and aortic wall velocity assessment, although the Doppler formula assumes continuous waves or long pulse lengths. By applying wide band transducers and short pulse lengths to achieve high spatial resolution, the usual Doppler formula assumptions are violated, and in particular the center frequency of the received echo will vary with depth. The presently-disclosed methods require both high temporal and spatial resolution and thus will take advantage of well-described analytical methods which provide estimates of the mean spatial frequency, mean temporal frequency, spatial bandwidth, and signal to noise ratio from which the velocity can be accurately determined. The presently-disclosed methods employ the complex cross-correlation model to estimate blood flow velocity and tissue motion by means of ultrasound. Among the advantages of the presently-disclosed methods are that they are independent of the bandwidth of RF ultrasound signals.

Fetal Aortic Cross Sectional Area Assessment

At the level of the time-averaged peak power of the near and far aortic wall reflections, the wall velocity is determined. The aortic wall is considered to be composed of different layers exhibiting different acoustic properties. The layers are adventitia, media and intima respectively. As the intima and the media have approximately the same acoustic impedances as one another, the transition between these two layers hardly results into a reflection. However, the adventitia-media and the intima-blood interfaces strongly reflect ultrasound at a perpendicular incidence angle. As these two reflections are not presented separately with the wavelengths needed in fetal or cardiac ultrasound scanning, decomposition of the aortic wall reflections is applied in order to discriminate between the different interfaces.

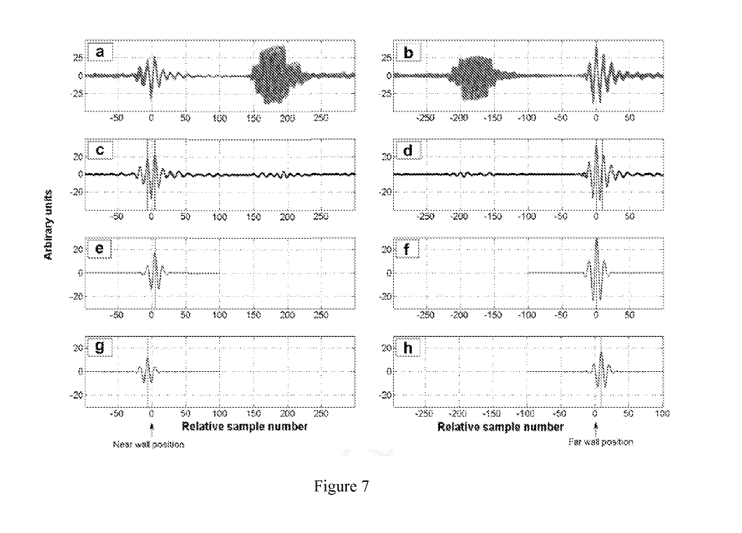

To achieve a mean estimate of the moving aortic wall reflections, the ultrasound RF data were repositioned relative to the previously tracked wall position. The tracked wall location was set to zero frame by frame, as shown in FIGS. 7a and 7b for the near wall and the far wall locations, respectively. The center frequency and the fractional bandwidth to allow description of the Gaussian pulse is determined using the results of the previously-mentioned complex cross correlation method.

In the decomposition model, the fetal aortic (or any other arterial) wall reflection is considered to be the sum of two Gaussian pulses, representing the adventitia-media and intima-blood interfaces. The decomposition method employs an iterative algorithm that uses the simplex search method. The seven largest extreme values of the mean wall reflection under consideration are determined and all possible combinations of these values, with their respective positions, are used to initialize the minimization search. From the absolute minimum of the searches, the intima media thickness (IMT), is defined as the distance between the two Gaussian pulses. Note that this is the mean IMT over the data acquisition period (e.g. two seconds or other time period). The wall thickness varies during the cardiac cycle and to obtain the dynamic IMT, the decomposition method is repeated for every wall reflection obtained during the data acquisition period using the tracked wall position and the mean wall thickness as initial values.

Subsequently, the distances between the near and far intima-blood interfaces are taken to represent the aortic lumen diameter, assuming that aorta has approximately circular symmetry, so that the cross sectional area of the fetal aorta can be calculated.

Pulse Wave Velocity (PWV)

An important aspect of performing fetal blood pressure measurements non-invasively by ultrasound is the assessment of wave propagation in the fetal aorta. Elastic vessels such as the fetal aorta and the pulmonary artery are close to purely elastic, i.e. visco-elastic contributions to the pressure-area relation are small. Therefore, this wave speed can be derived from the pressure waveform as well as the aortic cross-sectional area waveform, as both waves propagate at the same speed. Wave propagation implies that along a small segment of the fetal aorta (Δx) the cross-sectional area waveforms at the beginning and end of the segment differ by a transit time (Δt) and the pulse wave velocity (PWV) is defined as PWV=Δx/Δt.

The pulse wave velocity can be derived from the flow (q) and cross sectional area (A) waveform obtained at the same level of the fetal aorta. The multiplication of PWV by ΔA/ΔA provides:

$$PWV = \frac{Δ x}{Δ t} = \frac{Δ x \cdot Δ A}{Δ t \cdot Δ A} = \frac{Δ V}{Δ t \cdot Δ A} and with , \\ Δ q = \frac{Δ V}{Δ t} we can write \text{:} PMV = \frac{Δ q}{Δ A} .$$

The flow-area method to estimate pulse wave velocity is valid during the initial ejection phase of the heart, when reflections from impedance mismatches such as bifurcations are absent, implying that downstream reflected waves did not reach the measuring point for fetal aortic blood flow and cross sectional area. At the initial phase of ejection when the heart ejects a small volume (ΔV) in the aorta over a period (Δt) and the ejected volume is “accommodated” by the aorta by means of an increase of the cross sectional area (ΔA) over a certain length (Δx) of the aorta, the flow waveform is linearly related to the cross sectional area waveform and the slope of the straight portion of the flow area loop equals the pulse wave velocity. FIG. 4 shows the fetal aortic Flow-Area loop obtained in a human fetus. The advantage of applying the flow area method is that the pulse wave velocity is measured at a single spot (locally) and, therefore, can be applied early in pregnancy when sufficient aortic length is lacking. Pulse wave velocity assessment by the transit time method (Δx/Δt) is only possible at advanced gestational age when sufficient aortic length is available to determine the distance and transit time with sufficient accuracy.

Pulse Pressure Estimation

As the fetal aorta (or the pulmonary artery) is close to purely elastic, the relationship between the pulse wave velocity (PWV) and the pulse pressure (ΔP) can be described by the Bramwell-Hill equation.

$$PWV = \sqrt{\frac{\overline{A} \cdot Δ P}{ρ \cdot Δ A}} ,$$

with Ā=mean aortic cross sectional area, ΔA=Aortic area change and ρ=density of blood.

By setting the density of fetal blood to the generally accepted fixed value of 1.05 g/cm³ and expressing the area change (ΔA) as the deviation from the mean area (A (t)−Ā), the equation can be rewritten to scale the area waveform to the pulse pressure waveform (pₚ(t)) as,

$${p}_{p} ( t ) = 1.05 \frac{{PWV}^{2} ( A ( t ) - \overline{A} )}{\overline{A}}$$

Downstream Impedance Model

To describe the downstream impedance, the four element lumped-parameter model is applied. Arterial inertance is added to the three element model that combines the transmission line model with the classical two element Windkessel model such as shown in FIG. 5. In this model Rₚ represents the peripheral resistance and Cₐ the arterial compliance, (R_{c}) the characteristic resistance and Lₐ the arterial inertance. It is demonstrated that addition of the fourth element (inertance) improves compliance estimation and yields excellent shapes of pressure and flow. Moreover, all four parameters have their basis in arterial properties, for these reasons the four element model is selected for parameter estimation of vascular properties with emphasis on peripheral resistance and compliance.

Model Parameter Estimation

As in electrical circuits, the downstream impedance Z can be represented as:

$$Z = \frac{{R}_{c} i 2 π {fL}_{a}}{{R}_{c} + i 2 π {fL}_{a}} + \frac{{R}_{p}}{1 + i 2 π {fC}_{a} {R}_{p}}$$

where i represents the square root of −1 and f is the frequency.

From the detailed velocity and pulse pressure waveforms, the four parameters of the model can be estimated. The best estimates for the model parameters are found when the pulsatile part of the pressure as calculated from the product of blood flow and downstream impedance best fit with the actual pulse pressure as derived from the cross-sectional area waveform. The pressure waveform can be calculated in the frequency domain, using the discrete-time Fourier transform of q(t), given by:

$$Q ( f ) = {\sum }_{t = 0}^{T - 1} q ( t ) {e}^{- i2π ft}$$

where T is the total acquisition time, f is the frequency, and t is the time of a given timepoint. The pressure waveform p(t) in the time domain can be evaluated as the inverse Fourier transform of the product of the bloodflow waveform and the downstream impedance in the frequency domain. That is,

$$p ( t ) = \frac{1}{N} {\sum }_{f = - \frac{PRF}{2}}^{f = \frac{PRF}{2} - 1} Q ( f ) ( \frac{{R}_{c} i 2 π {fL}_{a}}{{R}_{c} + i 2 π {fL}_{a}} + \frac{{R}_{p}}{1 + i 2 π {fC}_{a} {R}_{p}} ) {e}^{i2π ft}$$

Where PRF is pulse repetition frequency (sampling frequency of the flow and area waveforms) and N is the number of samples. An iterative algorithm minimizes the sum of squared differences between the pulsatile part of the model calculated pressure waveform (pₚ(t)) and the pulse pressure as derived from the cross sectional area wawaveform (pₐ(t)). The function to be minimized is

$${F}_{min} = {\sum }_{t = 0}^{T - 1} {( {p}_{p} ( t ) - {p}_{a} ( t ) )}^{2}$$

This invention applies the “simplex search” method, generally referred to as unconstrained nonlinear optimization, to find this minimum. The initial value for Rₚ is calculated by taking the quotient of the RMS values of the pulsatile part of pulse-pressure and flow waveforms. The quotient of the stroke volume and the pulse pressure is taken as the initial value for Cₐ. Based on considerations from transmission line theory, the initial value of the characteristic resistance is taken as R_{c}=1.05*PWV/Ā and finally the initial inertance (L) is chosen in such a way that the absolute impedance (|i2πfL|) at the fetal heart rate is twice as high as the characteristic resistance.

Systolic, Diastolic, and Mean Pressure Estimation

The product of the mean blood flow and the estimated peripheral resistance provides the mean pressure. Note that the other model parameters, namely the characteristic impedance (Rc), the inertance (L), and the compliance (C), do not contribute to the mean pressure. From the sum of the mean pressure and the pulse pressure waveform, the systolic and diastolic blood pressure can be determined for every recorded heartbeat.

The following non-limiting Example is intended to be purely illustrative and is presented to show specific experiments that were carried out in accordance with embodiments of the invention:

### Example

The objective of the study described in this Example was to measure fetal aortic pulse wave velocity and lumen diameter waveforms and subsequently calculate local distensibility, compliance, and pulse pressure. A dedicated algorithm for optimizing lumen diameter assessment from radiofrequency ultrasound data is described. Biplane raw data were obtained from a matrix array transducer. We evaluated 83 confirmed, normally-developing pregnancies at 22-38 wk. Fetal aortic pulse wave velocity is calculated as (PWV, m/s)=0.047×gestational age (wk)+1.241, and the distensibility coefficient is calculated as (1/kPa)=1/(1.04×PWV²). The logarithm of the local compliance index (mm²/kPa) and the pulse pressure (kPa) were both linearly related to gestational age as 0.022×GA (wk)−0.343 and 0.012×GA (wk)+0.931, respectively. Thus, fetal aortic elastic properties can be derived from phase-sensitive radiofrequency data and multiline diameter assessment.

In the Example below, only fetal aortic diameter measurements are obtained, in order to calculate pulse wave velocity and pulse pressure, but not systolic and diastolic fetal blood pressures. As the transit time method is applied to measure the pulse wave velocity, the methods employed in the Example cannot be applied early in pregnancy when sufficient aortic length is lacking to measure the transit time. Thus, using a conventional ultrasound systems (in this case supplemented with a raw RF data interface which is not a standard feature of this particular system) in advanced pregnancies, the pulse pressure can be measured but not the systolic and diastolic pressure. Nevertheless, the Example shows that one can apply the disclosed decomposition method on fetal aortic data and that one can measure pulse pressure in the human fetus.

Fetuses born small for gestational age are likely to be predisposed to atherosclerotic cardiovascular disease in later life. This might result from alterations in the elastic properties of the fetal aorta. Impaired synthesis of elastin during the period of rapid fetal growth has been hypothesized as an initiating event in the pathogenesis of systemic hypertension. Observation of diameter waveforms in the fetal inferior vena cava and the fetal aorta have revealed hemodynamic changes between normal and compromised pregnancies. As the elastic properties of the vessel wall are unknown in these studies, whether the observed differences between the studied groups originate from pressure or elastic changes, or a combination, also remains unknown. It has been hypothesized that the changes in scleroprotein structure and augmented wall thickness they observed in neonates may have been caused by umbilical placental insufficiency, hypertension and increased afterload in fetal life. Measurement of elastic aortic wall properties such as distensibility and compliance in the fetus would benefit our understanding of the pathophysiologic process. The small size of the human fetus, approximately 300 g at mid-gestation and 3500 g at term, poses a challenge to the determination of elastic aortic wall properties from multiple aortic diameter and pulse wave velocity (PWV) measurements. Moreover, the center frequency that can be applied is relatively low, as penetration depth is needed for fetal ultrasound scanning, and therefore, spatial resolution is limited.

To this end, first we derived longitudinal and cross sectional (biplane) data from the fetal aorta at the level of the diaphragm. Second, we developed an algorithm to optimize lumen diameter measurements by decomposing the ultrasound fetal aortic wall reflection in the radiofrequency (RF) data into two Gaussian pulses. Third, we calculated the distensibility, compliance and pulse pressure of the fetal aorta. Finally, we assessed the reliability of the method by analyzing intra- and inter-observer variability.

This Example describes the methods used to make longitudinal and cross-sectional ultrasound measurements for reliable estimation of fetal aortic lumen diameter and pulse wave velocity and measurements of diameter waveforms for subsequent estimates of local Q4 distensibility, compliance and pulse pressure.

Methods

Patients

We studied 121 healthy pregnant women whose fetuses varied in gestational age (GA) from 22 to 38 wk, over the period May 2008 to January 2009. The women were recruited from patients at the outpatient clinic of the Department of Maternal-Fetal Medicine of Radboud University Nijmegen Medical Centre (The Netherlands). The study protocol was approved by the hospital ethics committee and written informed consent was obtained from all participating women. Pregnancy was classified as normal if it was uncomplicated and resulted in the delivery of a healthy child at ≧37.0 wk, with a birth weight between the 5th and 95th percentile reference lines of the Dutch population (Visser et al. 2009). We excluded 28 data sets because of incomplete data (n=19), premature delivery (n=2), hypertensive disease (n=3), low birth weight (n=2) and macrosomia (n=2). The remaining data sets of 83 women with normal pregnancies were used for further analysis.

Ultrasound Recordings

Ultrasound RF data were acquired from the descending fetal aorta at the level of the diaphragm using an iE33 ultrasound system (Philips Medical Systems, Bothell, Wash., USA), equipped with an X7 matrix array ultrasound transducer (bandwidth: 2-7 MHz) and a custom-designed RF interface (sampling frequency: 32 MHz) in biplane mode. All data sets were acquired using the same instrument settings. Acquisition time was set at 2 s, the sector angle to scan the longitudinal section of the fetal aorta was set at 30° (40 image lines) and the cross-sectional sector angle was set at 27° (36 image lines). The perpendicular and longitudinal planes shared the same center line. By defining a “frame” as a set consisting of the simultaneously derived longitudinal and the cross sectional fetal aortic planes, data frames were obtained at the rate of 82 Hz.

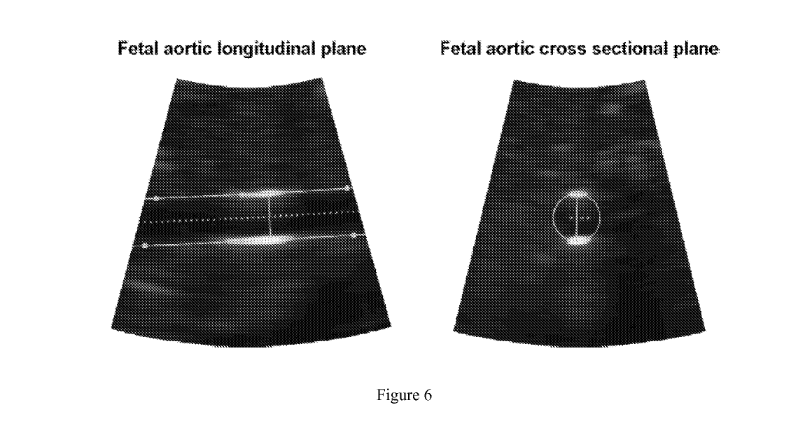

Radiofrequency data were transmitted to a USB mass storage device for offline analysis. Data were analyzed with the use of MATLAB software (Version 7.5 Release 2007B, The Mathworks, Natick, Mass., USA). An analytic representation of the RF data was derived using the Hilbert transform method and was used for visualizing the echograms after log compression. FIG. 6 is a zoomed example of the longitudinal and cross-sectional planes of the descending fetal aorta. Additional figures employed to illustrate the techniques in this Example are for the same fetus.

Localization of the Near and Far Fetal Aortic Walls

The time-averaged positions of the moving near and far fetal aortic walls were identified manually. To this end, an intensity-averaged image was constructed, derived from all 164 frames collected in the 2-s acquisition period (FIG. 6). Subsequently, at least three different positions along the near and far aortic walls were marked by mouse clicking. A second-degree polynomial curve was calculated through these marker points to identify the mean positions of the near and far walls, respectively.

Fetal Aortic Diameter Waveforms

Fetal aortic wall displacement was measured from every scan line. A 2-mm window (85 RF data points) was positioned automatically on the crossings of each scan line and the aforementioned curved lines describing the approximate near and far aortic walls. The displacement between two successive aortic wall reflections within each window was determined by echo tracking through cross-correlation.

FIG. 6 shows an intensity-averaged image from all 164 frames collected in the 2-s acquisition period. The three dots on the near and far fetal aortic wall presentation are manually set. A second-degree polynomial curve is calculated through the three marker points to represent the approximate time-averaged center of the moving near and far aortic walls. The dotted line represents the center of the aortic lumen. It is assumed that in the longitudinal as well as the cross-sectional plane, maximum intensity is achieved at an angle of incidence of the ultrasound beam perpendicular to the fetal aortic wall. Perpendicular insonation with the aortic walls is marked by the lines connecting the near and far walls, representing the approximate time averaged aortic diameter. It is assumed that the aorta has an approximately circular cross section which is presented as a circle, because the reflection is dependent on the angle between the aortic wall and the incident beam. Nonetheless, the circular cross-sectional area cannot be clearly observed in the cross-sectional image.

Pulse Wave Velocity

Fetal aortic pulse wave velocity was determined from the diameter waveforms derived from the longitudinal plane. The length along which the descending aorta can be visualized depends on fetal position and fetal size. Shadowing from the ribs and the anisotropic behavior of ultrasound might further limit the number of diameter waveforms eligible for pulse wave velocity assessment. For this purpose, echo tracking waveforms from all longitudinal lines were obtained, and only the waveforms with the typical fetal aortic shape were manually selected for further analysis.

Pulse wave velocity was calculated as the reciprocal of the slope of the regression line of mean transit times over the number of heartbeats and the distance the wave propagated along the aortic segment. Transit times of the onset of systolic diameter waveforms were determined by the tangent method. Transit times were corrected for the scanning sequence and for the distance from the ultrasound transducer to the center of the aortic lumen. The mean transit time for all cardiac cycles within the 2-s scan period was used in the calculation of pulse wave velocity. The distance the wave propagated was determined along the center of the aorta as the cumulative sum of the distances.

Aortic Lumen Diameter

The diameter waveform exhibiting maximum wall reflections was assumed to be perpendicularly insonated by the ultrasonic beam and was selected automatically in the cross-sectional and longitudinal planes. The aortic lumen diameter was defined as the distance between the intima-blood interfaces. The interfaces were determined using the aortic wall model as reported by Wikstrand (2007). Because the reflections of the adventitia-media, media-intima, and intima-blood interfaces are not presented separately with the wavelengths needed in fetal ultrasound scanning, the aortic wall reflections had to be decomposed to discriminate between the different interfaces.

To achieve a mean estimate of the moving aortic wall reflections, the ultrasound RF data were repositioned relative to the previously tracked wall position. The tracked wall location was set to zero frame by frame, as indicated in FIGS. 7a and 7b for the near wall and far wall locations, respectively. The center frequency and fractional bandwidth were experimentally determined as 2.74 MHz and 55% to allow description of the Gaussian pulse.

FIGS. 7a-7h show tracked aortic wall positions for data collected from a human fetus. In FIG. 7a, the tracked near-wall reflection is set to zero on a frame-by-frame basis. The value zero on the horizontal axis represents the initial tracking position, that is, the center of the fixed cross-correlation window. FIG. 7b shows similar data from the same sample in which the tracked far-wall reflection is set to zero on a frame-by-frame basis. FIGS. 7c and 7d show overlaid data plots which indicate that the data points that do not move synchronously with the fetal aortic wall are spread out and consequently, the time-averaged waveform shows a detailed mean wall reflection according to near- or far-wall tracking. FIGS. 7e-7h show that the mean wall reflections are decomposed into separate Gaussian pulses to distinguish reflections from the media (intima)-blood interface (FIGS. 7e, 7f) and from the adventitia-media interface (FIGS. 7g, 7h). The vertical (red) lines mark the positions of the interfaces between the different layers. Note that in FIGS. 7c and 7d, the reflections from different layers are not recognizable to the naked eye; the black dots represent the averaged radiofrequency data points, and the (red) trace represents the best fit of the sum of two Gaussian pulses through these data points.

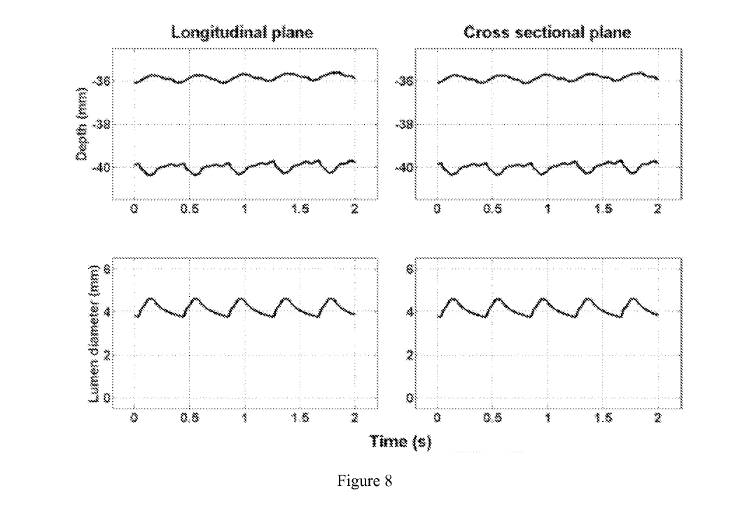

In the decomposition model, the fetal aortic wall reflection was considered to be the sum of two Gaussian pulses representing the adventitia-media and intima-blood interfaces, respectively. Because the intima and media have approximately the same acoustic impedance, the transition between these two layers hardly results in a reflection. The decomposition method employed an iterative algorithm that uses the simplex search method. The seven largest extreme values of the mean wall reflection under consideration were determined and all possible combinations of these values, with their respective positions, were used to initialize the minimization search. From the absolute minimum of the searches, the position of the intima-blood interface was determined, expressed relative to the manually-selected position. The distance between the near and far intima-blood interfaces was taken to represent aortic lumen diameter (FIG. 8).

The top left and right panels of FIG. 8 indicate the positions of the intima (media)-blood interface frame by frame, relative to the ultrasound transducer face, along the scan lines that exhibit maximum wall reflections for the longitudinal and cross-sectional planes, respectively. The bottom left and right panels of FIG. 8 indicate the distance between these far- and near-wall interfaces representing the fetal aortic lumen diameter waveform as derived from the longitudinal and cross-sectional planes.

Distensibility, Compliance and Pulse Pressure

Peak systolic and end-diastolic lumen diameters were determined from the aortic diameter waveform. Distensibility, compliance and pulse pressure of the fetal aorta were calculated from these diameters and pulse wave velocity, as detailed below in the Appendix.

Intra- and Inter-Observer Variability

To investigate the reliability of user intervention on pulse wave velocity measurements, that is (i) marking the near and far aortic walls by mouse clicking and (ii) manually selecting diameter waveforms, this procedure was performed twice by two observers.

Statistical Analysis

All statistical analyses were performed using the SPSS Statistical Package, Release 16.0 (SPSS, Chicago, Ill., USA). The agreement between the aortic lumen diameters derived from the longitudinal and cross-sectional planes was assessed by Bland-Altman analysis (Bland and Altman 1999). Age-related reference percentiles were calculated as described by Altman (1993). The intra- and inter-observer variability of pulse wave velocity measurement is expressed as the coefficient of variation (CV), which is defined as the ratio of the standard deviation of differences between repeated pulse wave velocity measurements to the mean of the measurements over all patients. Moreover, the limits of agreement were calculated by Bland-Altman analysis. The level of statistical significance was set at 0.05.

Results

The number of confirmed normally developing infants was 83. Gestational age at the time of examination ranged from 22 4/7 to 38 3/7 wk. The numbers of fetuses per completed week of gestation were: 22 (4), 23 (4), 24 (1), 25 (7), 26 (3), 27 (7), 28 (4), 29 (8), 30 (7), 31 (8), 32 (9), 33 (5), 34 (5), 35 (4), 36 (3), 37 (1), 38 (3). The depth at which the fetal aorta could be obtained ranged from 4 to 9 cm; the distance along the descending part of the aorta from which pulse wave velocity was derived ranged from 7.5 to 38 mm.

Fetal Aortic Diameter

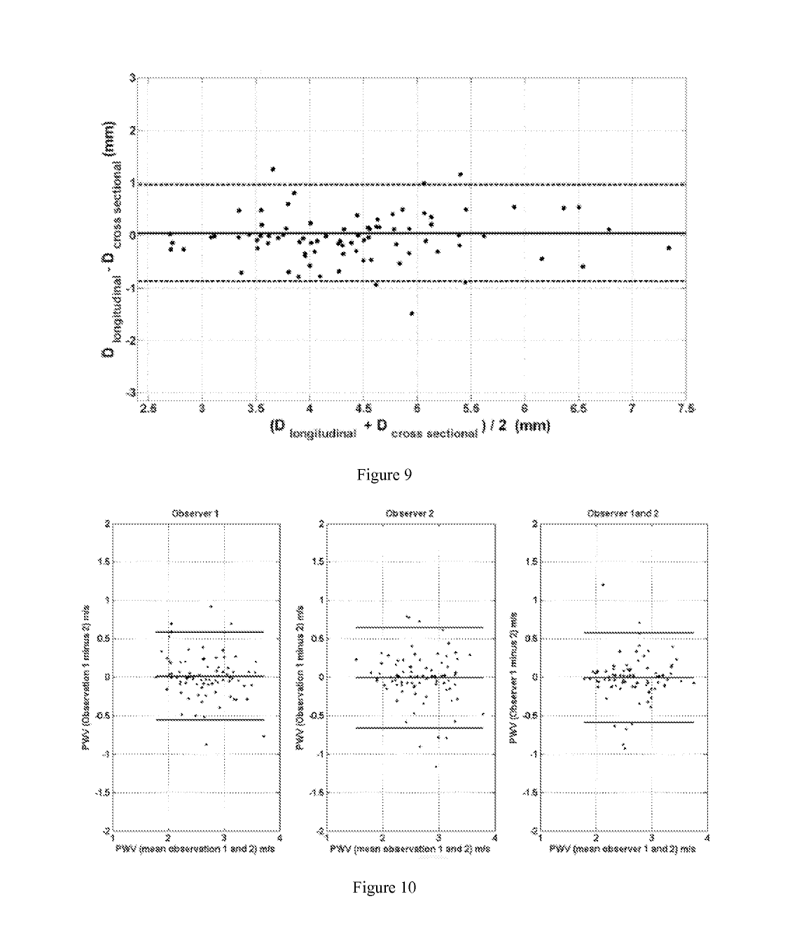

Fetal aortic end-diastolic diameters measured in the longitudinal and cross-sectional planes did not differ significantly, as illustrated in FIG. 9 (mean difference=0.038 mm, p=0.45). The standard deviation of the differences was 0.457 mm. Further analyses were performed on longitudinally measured values only, as these did not differ from the cross-sectional data and longitudinal scanning is the method commonly used for fetal aortic examinations.

The estimated mean aortic lumen diameter was 4.7% lower than the manually determined diameter (p<0.001), with the mean of the paired differences equal to 0.189±0.324 mm (standard deviation [SD]).

Fetal Aortic Pulse Wave Velocity

The transit time-distance relationship was a good linear fit, with an interquartile range for the mean squared errors of 0.20-0.75. The interquartile range for the explained variances (R2) of the linear fit associated with each subject was 91%-97%.

Pulse Wave Velocity: Intra- and Inter-Observer Variability

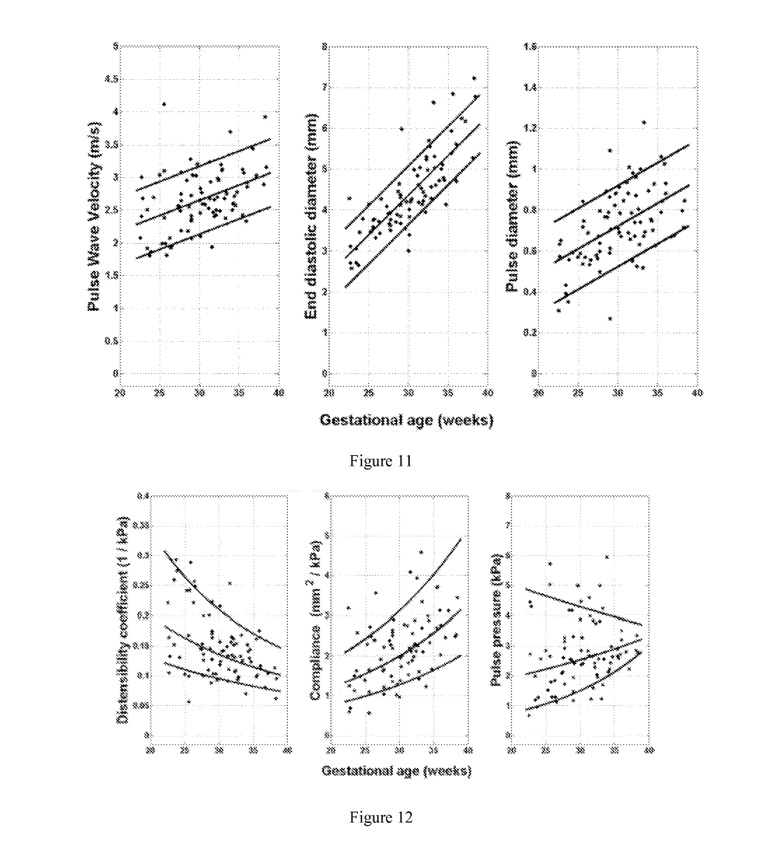

Intra- and inter-observer variability of pulse wave velocity assessment, expressed as the coefficient of variation, was low. Intra-observer variation was 10.8% and 12.4%, respectively, for observers 1 and 2. The mean difference between observations was small: 0.01 m/s (SD=0.286, p=0.77) and −0.01 m/s (SD=0.328, p=0.76), respectively, for observers 1 and 2. Inter-observer variability was 11%, and the difference between observers was small: −0.01 m/s (SD=0.292, p=0.73). Bland-Altman analyses revealed no relationship between variability and magnitude of the measured pulse wave velocity, as seen in FIG. 10. The correlation coefficients between observations were R=0.828 (p<0.001) and R=0.771 (p<0.001) for observers 1 and 2, respectively, and R=0.796 (p<0.001) between observers.

Fetal Pulse Wave Velocity, End-Diastolic Lumen Diameter and Pulse Diameter

Pulse wave velocity, end-diastolic lumen diameter and pulse diameter all increased linearly with gestational age, as outlined in Table 1 and illustrated in FIG. 11, respectively. The variance of the residuals was independent of gestational age.

TABLE 1
Fetal aortic parameters expressed as a function of gestational age (in the second half of gestation)

| | | p-value | | p-value |
|---|---|---|---|---|
| | Equation | of slope | SD residual | of slope |
| Parameters | | | | |
| Pulse wave velocity (m/s) | 0.047 × GA + 1.251 | <0.001 | 0.419 | — |
| End-diastolic diameter (mm) | 0.194 × GA − 1.448 | <0.001 | 0.587 | — |
| Pulse diameter (mm) | 0.022 × GA + 0.052 | <0.001 | 0.154 | — |
| Derived variables | | | | |
| Distensibility coefficient, D_{c} = 1/(1.04 × | — | — | — | — |
| PWV²) (l/kPa) | | | | |
| Log compliance coefficient (mm²/kPa) | 0.022 × GA − 0.343 | <0.001 | 0.159 | — |
| Log pulse pressure (kPa) | 0.012 × GA + 0.931 | 0.022 | −0.015 × GA + 0.614 | <0.001 |

GA = gestational age;
PWV = pulse wave velocity.

FIG. 12 shows the calculated distensibility coefficient (left), local fetal aortic compliance (center), and pulse pressure (right) with superimposed 10th, 50th and 90th percentile lines. Because fetal aortic distensibility is inversely related to the squared pulse-wave velocity, the percentile lines are calculated from the pulse wave velocity linear regression results. The local compliance coefficient was linearly related to gestational age in the log domain, and the variance of the residuals was independent of gestational age, resulting in curved, monotonically increasing lines with gestational age on a linear compliance scale. The pulse pressure had a log-linear relationship with gestational age, and the absolute residuals in the log domain varied linearly with gestational age.

The ability to store phase-sensitive RF data provides the opportunity to investigate the fetal circulation in more detail than previously possible. In this Example, we determined the feasibility of measuring fetal aortic distension waveforms from multiple ultrasound scan lines and deriving the pulse wave velocity from these data. Subsequently, we calculated the pulse pressure and two fetal aortic elastic properties, the distensibility coefficient and compliance. The ability to estimate these parameters and to analyze them may provide new insight into the physiology of the developing fetus.

Decomposition of aortic wall reflections solves a problem of limited spatial resolution in obstetric ultrasound. Obstetric scanning systems typically facilitate measurements up to 20 cm deep, to cover the widely varying range of fetal aortic presentation. This implicates relatively long ultrasound wavelength and poor resolution. Separate reflections from the adventitia-media and intima-blood interfaces could not be visually recognized either from the RF or demodulated data because of the overlap between the two reflected pulses. However, they could be discriminated after decomposition. The blood-media transition was better defined than the adventitia-media transition, because the wall reflection was larger and the ultrasound contrast was more pronounced in the former as the echogenicity of blood is less than that of the aortic wall. As a result, fetal aortic lumen diameter could be determined accurately.

Fetal Aortic Lumen Assessment

The procedure of manually marking the near and far aortic walls by mouse clicking did not have an effect on the automatic selection of the line that exhibits maximum wall reflection (perpendicular incidence angle with the aortic wall) in the fetal aortic longitudinal and cross-sectional scan planes, respectively. The decomposition method for aortic lumen assessment along these lines also was not affected, indicating that the algorithm correctly compensates for different initial values introduced by different users or repeated marking of the aortic wall. The algorithms for diameter assessment correctly compensated for slightly different initial values. For determination of systolic and diastolic diameters from ultrasound 2-D images, intra- and inter-observer coefficients of variation of 5.4% and 7.7%, respectively, have been reported. The algorithm described in this Example compensates for this type of error.

The fetal aortic lumen diameter from decomposition of the wall reflections was, on average, 4.7% smaller than the diameter determined from manual cursor placement in the longitudinal presentation. Intuitively, one would mark the spot exhibiting the highest intensity as indicating the aortic wall. This is more likely to be the average position of the intima-media than the position of the intima-blood interface and would thereby overestimate aortic lumen diameter. The decomposition method systematically searches for intima-blood interfaces to determine the lumen of the aorta, which explains the systematically smaller aortic diameter compared with the diameter derived from manual curser placement.

The mean fetal aortic lumen diameters as determined in the longitudinal and cross-sectional planes were virtually identical, as seen in FIG. 9. The differences between the measurements in the longitudinal and cross-sectional planes were independent of the size of the diameter, and not significantly different from zero. This implies that the longitudinal and cross-sectional measured aortic diameters can be used interchangeably. The limits of agreement were as high as ±0.9 mm. The variance between the measurements can partly be explained by the distances between the automatically selected ultrasound lines in the longitudinal and cross-sectional planes and the downstream tapering shape of the fetal aorta. Off-plane scanning of the longitudinal aorta is another possible explanation of the observed variance. Our results may be less accurate than technically feasible, because we performed our measurements offline. More accurate results might have been obtained if the sonographer would have had available dedicated ultrasound equipment that would allow examination of fetal wall reflections during scanning and instantaneous adjustment of the scan plane according to a pre-defined set of criteria. Such a system would be analogous to that reported for carotid artery layer localization with non-invasive ultrasound in adults.

Fetal Aortic Pulse Wave Velocity Assessment

The best performance in wall tracking is obtained when wall reflections as obtained from the automatically selected echo lines in the longitudinal and cross-sectional planes are maximal. Because of the anisotropic behavior of ultrasound, it is not clear from the B-mode image which part of the aorta is eligible for pulse wave velocity analysis. Alternatively, the first and last diameter waveforms are selected manually by comparing the wave shape of individual waveforms with the wave shape at maximum wall reflections. The fetal aorta was scanned at the level of the diaphragm, which implies that the longitudinal scan represents a part of the thoracic aorta as well as the abdominal aorta. The thoracic part might be partly invisible because of shadowing from the ribs, which might cause failure of echo wall tracking between the first and last selected ultrasound echo lines. The diameter waveforms affected by shadowing were de-selected. As there are no strict criteria to accept or reject waveforms, the set of waveforms will vary between observers and within observers for repeated selection procedures. Bland-Altman analysis revealed that the variability introduced by this procedure into pulse wave velocity outcome is considerable. The pulse wave velocity varies along the descending aorta and increases in the downstream direction. Moreover, to perform well, the tangent method needs a reflection-free period of the cardiac cycle. If waveforms are obtained near bifurcations or near the bifurcation into the femoral arteries, reflections might be present early in the systolic phase of the cardiac cycle, adversely affecting pulse wave velocity assessment. This might be unavoidable at mid-gestation when the fetus is small, because sufficient aortic length is needed to determine pulse wave velocity. Shadowing of the ribs is less dominant early in pregnancy, but insufficient aortic length and reflections from near bifurcations might influence the accuracy of pulse wave velocity assessment. In advanced pregnancies, the exact position and shadowing from the ribs might become dominant and influence the accuracy of pulse wave velocity assessment.

The mean differences within and between observers were not different from zero; neither was the variability influenced by the magnitude of the pulse wave velocity, implying that the described method can be used for epidemiologic studies.

Fetal Aortic Wall Properties

Fetal aortic pulse wave velocity increases linearly with gestational age from 22 to 38 wk gestation, from 2.29±0.4 to 3.04±0.4 m/s. Similar fetal aortic pulse wave velocities were reported in previous studies from uncomplicated pregnancies. Much higher values (5.2±0.8 m/s) have been reported in young adults and the elderly aged >80 (14.2±4.8 m/s), demonstrating that pulse wave velocity increases many-fold as distensibility decreases with age from intrauterine life to old age.

In normal fetal life, the aorta matches the physiologic adaptations of growth, which include increased cardiac output and reduced vascular resistance. From 22 to 38 wk, fetal aortic compliance increases by 125% and pulse pressure increases by 36% as a result of the increasing aortic dimensions, despite a 43% reduction in distensibility. This process continues into healthy adulthood.

In some adults, pathophysiologic processes may result in early abnormal stiffening of the aortic wall. This may result in increased systolic blood pressure, increased pulse pressure and increased pulse wave velocity, while diastolic blood pressure remains relatively unaffected. The theory of fetal origins of adult disease suggests that small changes from the norm in fetal life will result in higher susceptibility to vascular disease in later life. Accurate tools are needed to characterize such variations to verify this theory experimentally. We found that fetal aortic pulse wave velocity, distensibility, compliance and pulse pressure can be measured or calculated. The observed wide range between the 10th and 90th percentile lines for fetal aortic elastic wall properties indicates that large studies are needed to determine differences between normal and compromised pregnancies.

Fetal aortic wall properties can be derived from phase-sensitive radiofrequency data and multi-line diameter assessment. A statistically significant increase is found for fetal aortic pulse wave velocity, compliance and pulse pressure during the second half of pregnancy.

### APPENDIX

The relationship between pulse wave velocity (PWV) and pulse pressure (AP) in an elastic thin-walled tube containing incompressible fluid was first described by Isaac Newton (1643-1727) and Thomas Young (1773-1829). Later it became known as the Bramwell-Hill equation (Bramwell and Hill 1922). When this theory is applied to aortic blood flow in the absence of reflections, the relationship is

$$PWV = \sqrt{\frac{V \cdot Δ P}{ρ \cdot Δ V}} [ m \text{/} s ] \qquad ( A1 )$$

where ρ is the density of blood and is considered constant at 1040 kg/m³, V is the end-diastolic volume of the aortic segment under consideration and ΔV is the change in volume from the end-diastolic to peak systolic phases of the cardiac cycle. Equation (A1) can be applied to derive the pulse pressure, distensibility coefficient and fetal aortic compliance from multi-line fetal aortic diameter measurements, provided accurate estimates of pulse wave velocity and fetal aortic dimensions can be made.

Given the generally accepted model assumptions that (i) the fetal aorta is tethered by an axial constraint and, therefore, the length of the aorta does not change during the cardiac cycle, and (ii) the descending aorta is close to purely elastic, that is, visco-elastic contributions to the pressure-area relationship are small, the ratio ΔV/V can be rewritten as ΔA/A, where A is the local cross-sectional area of the fetal aorta. Assuming circular symmetry, area can be replaced by πd²/4, where d is the lumen diameter of the aorta, and eqn (A1) can be rewritten to solve for pulse pressure ΔP as

$$Δ P = \frac{ρ \cdot ( {d}_{ps}^{2} - {d}_{ed}^{2} ) \cdot {( PWV )}^{2}}{{d}_{ed}^{2}} [ Pa ] \qquad ( A2 )$$

where dₚₛ is peak systolic diameter, and d_{ed} is end-diastolic diameter. The distensibility coefficient, a relative measure that characterizes the elastic behavior of the fetal aorta, is defined as D_{c}=(ΔV/V)/ΔP, and can be related to pulse wave velocity using (A1) as

$${D}_{c} = \frac{Δ V / V}{Δ P} = \frac{1}{ρ \cdot {( PWV )}^{2}} [ {Pa}^{- 1} ] \qquad ( A3 )$$

The fetal aortic compliance coefficient (Cc), defined as ΔA/ΔP, can be interpreted as the compliance per unit length. It follows from (A1) that

$${C}_{c} = \frac{Δ A}{Δ P} = \frac{π \cdot {d}_{ed}^{2} / 4}{ρ \cdot {( PWV )}^{2}} [ {m}^{2} \cdot {Pa}^{- 1} ] \qquad ( A4 )$$

### REFERENCES

Each of the following is incorporated herein by reference in its entirety:
Akira M, Yoshiyuki S. Placental circulation, fetal growth, and stiffness of the abdominal aorta in newborn infants. J Pediatr 2006; 148(1):49-53.
Altman D G. Construction of age-related reference centiles using absolute residuals. Stat Med 1993; 12(10):917-924.
Avolio A P, Butlin M, Walsh A. Arterial blood pressure measurement and pulse wave analysis—their role in enhancing cardiovascular assessment. Physiol Meas 2010; 31:R1-47.
Barker D J. Intrauterine programming of coronary heart disease and stroke. Acta Paediatr Suppl 1997; 423:178-182.
Benetos A, Buatois S, Salvi P, Marino F, Toulza O, Dubail D, Manckoundia P, Valbusa F, Rolland Y, Hanon O, Gautier S, Miljkovic D, Guillemin F, Zamboni M, Labat C, Perret-Guillaume C. Blood pressure and pulse wave velocity values in the institutionalized elderly aged 80 and over: baseline of the PARTAGE study. J Hypertens 2010; 28:41-50.
Benetos A, Salvi P, Lacolley P. Blood pressure regulation during the aging process: the end of the ‘hypertension era’? J Hypertens 2011; 29(4):646-652.
Bland J M, Altman D G. Measuring agreement in method comparison studies. Stat Methods Med Res 1999; 8(2):135-160.
Brands P J, Hoeks A P, Ledoux L A, Reneman R S. A radio frequency domain complex cross-correlation model to estimate blood flow velocity and tissue motion by means of ultrasound. Ultrasound Med Biol. 1997; 23(6):911-20
Brands P J, Willigers J M, Ledoux L A, Reneman R S, Hoeks A P. A noninvasive method to estimate pulse wave velocity in arteries locally by means of ultrasound. Ultrasound Med Biol 1998; 24(9):1325-1335.
Gardiner H, Brodszki J, Marsal K. Ventriculovascular physiology of the growth-restricted fetus. Ultrasound Obstet Gynecol 2001; 18:47-53.
Gardiner H M. Intrauterine programming of the cardiovascular system. Ultrasound Obstet Gynecol 2008; 32(4):481-484.
Hermeling E, Reesink K D, Reneman R S, Hoeks A P. Measurement of local pulse wave velocity: effects of signal processing on precision. Ultrasound Med Biol 2007; 33(5):774-781.
Hille E T, Weisglas-Kuperus N, van Goudoever J B, Jacobusse G W et al. Dutch Collaborative POPS 19 Study Group. Functional outcomes and participation in young adulthood for very preterm and very low birth weight infants: the Dutch Project on Preterm and Small for Gestational Age Infants at 19 years of age. Pediatrics. 2007 September; 120(3):e587-95.
Hu J, Bjorklund A, Nyman M, Gennser G. Mechanical Properties of Large Arteries in Mother and Fetus during Normal and Diabetic Pregnancy. J Matern Fetal Investig 1998; 8(4):185-193.
Hu J, Nijhuis I J, ten Hof J, Gennser G. Dependence of aortic pulse wave assessments on behavioural state in normal term fetus. Early Hum Dev 1997; 48:59-70.
Kyrklund-Blomberg N B, Hu J, Gennser G. Chronic effects of maternal smoking on pulse waves in the fetal aorta. J Matern Fetal Neonatal Med 2006; 19:495-501.
Lagarias J C, Reeds J A, Wright M H, Wright P E. Convergence Properties of the Nelder-Mead Simplex Method in Low Dimensions. SIAM Journal of Optimization 1998; 9:12-147.
Marple S L. Computing the discrete-time analytic signal via FFT. IEEE Transactions on Signal Processing 1999; 47(9):2600-2603.
Martyn C N, Greenwald S E. Impaired synthesis of elastin in walls of aorta and large conduit arteries during early development as an initiating event in pathogenesis of systemic hypertension. Lancet 1997; 350:953-955.
Moddemeijer R. On the Determination of the Position of Extrema of Sampled Correlator. IEEE Transactions on Acoustics, Speech, and Signal Processing 1991; 3(1):216-218.
Mori A, Trudinger B, Mori R, Reed V, Takeda Y. The fetal central venous pressure waveform in normal pregnancy and in umbilical placental insufficiency. Am J Obstet Gynecol 1995; 172(1 Pt 1):51-57.
Mori A, Trudinger B, Mori R, Reed V, Takeda Y. The fetal aortic pressure pulse waveform in normal and compromised pregnancy. Br J Obstet Gynaecol 1997; 104(11):1255-1261.
Mori A, Iwabuchi M, Makino T. Fetal haemodynamic changes in fetuses during fetal development evaluated by arterial pressure pulse and blood flow velocity waveforms. BJOG 2000; 107(5):669-677.
Reusz G S, Cseprekal O, Temmar M, Kis E, Cherif A B, Thaleb A, Fekete A, Szabó A J, Benetos A, Salvi P. Reference values of pulse wave velocity in healthy children and teenagers. Hypertension 2010; 56(2):217-224.
Rossi A C, Brands P J, Hoeks A P. Automatic localization of intimal and adventitial carotid artery layers with noninvasive ultrasound: a novel algorithm providing scan quality control. Ultrasound Med Biol 2010; 36(3):467-479.
Sindberg Eriksen P, Gennser G, Lindstr [symbol] om K, Benthin M, Dahl P. Pulse wave recording—development of a method for investigating foetal circulation in utero. J Med Eng Technol 1985; 9(1):18-27.
Stergiopulos N, Westerhof B E, Westerhof N. Total arterial inertance as the fourth element of the windkessel model. Am J Physiol. 1999 January; 276(1 Pt 2):H81-8
Struijk P C, Mathews V J, Loupas T, Stewart P A, Clark E B, Steegers E A, Wladimiroff J W. Blood pressure estimation in the human fetal descending aorta. Ultrasound Obstet Gynecol. 2008 October; 32(5):673-81
Struijk P C, Wladimiroff J W, Hop W C, Simonazzi E. Pulse pressure assessment in the human fetal descending aorta. Ultrasound Med Biol 1992; 18(1):39-43.
Visser G H, Eilers P H, Elferink-Stinkens P M, Merkus H M, Wit J M. New Dutch reference curves for birthweight by gestational age. Early Hum Dev 2009; 85(12):737-744.
Wikstrand J. Methodological considerations of ultrasound measurement of carotid artery intima-media thickness and lumen diameter. Clin Physiol Funct Imaging 2007; 27(6):341-345.
Womersley J R. Oscillatory flow in arteries: the constrained elastic tube as a model of arterial flow and pulse transmission. Phys Med Biol 1957; 2:178-187.

Various features and advantages of the invention are set forth in the following claims.

1. An ultrasound transducer system, comprising:
at least three ultrasound transducer arrays including a central transducer array and at least two lateral transducer arrays located adjacent the central transducer array, wherein the at least three ultrasound transducer arrays are arranged such that ultrasound beam paths of the at least three ultrasound transducer arrays overlap in an approximately planar location.

2. The ultrasound transducer system of claim 1, further comprising a controller in communication with the at least three ultrasound transducer arrays, wherein the controller is configured to obtain a two-dimensional image from within a subject using at least one of the at least three ultrasound transducer arrays.

3. The ultrasound transducer system of claim 2, further comprising a display in communication with the controller, wherein the controller is further configured to display the two-dimensional image on the display.

4. The ultrasound transducer system of claim 3, further comprising a user input in communication with the controller, wherein the controller is further configured to obtain a location within the subject using the user input, wherein the location indicates a center of a lumen of an aorta.

5. The ultrasound transducer system of claim 4, wherein the controller is further configured to generate a center array echo line from the central transducer array and a plurality of side array echo lines from each of the lateral transducer arrays, wherein each of the center array echo lines and the side array echo lines cross at the location.

6. The ultrasound transducer system of claim 5, wherein the controller is further configured to use electronic beam steering to generate the center array echo line and the plurality of side array echo lines.

7. The ultrasound transducer system of claim 5, wherein the controller is further configured to obtain ultrasound data from each of the center array echo line and the plurality of side array echo lines.

8. The ultrasound transducer system of claim 7, wherein the controller is further configured to obtain ultrasound data for a period of at least two seconds.

9. The ultrasound transducer system of claim 7, wherein the controller is further configured to obtain arterial blood flow and diameter waveforms at a sampling rate of between 80 Hz and 200 Hz.

10. The ultrasound transducer system of claim 7, wherein the controller is further configured to calculate at least one of heart rate, aortic wall thickness, time averaged aortic lumen diameter, pulse wave velocity, local aortic distensibility coefficient, aortic compliance coefficient, elastic modulus of the aortic wall, mean aortic blood flow, stroke volume, downstream peripheral resistance, compliance of the fetal vascular bed, systolic aortic blood pressure, diastolic aortic blood pressure, and mean aortic blood pressure based on the ultrasound data.

11. An ultrasound transducer, comprising:
at least three ultrasound transducer arrays including a central transducer array and at least two lateral transducer arrays located adjacent the central transducer array, wherein the at least three ultrasound transducer arrays are arranged such that ultrasound beam paths of the at least three ultrasound transducer arrays overlap in an approximately planar location.

12. The ultrasound transducer of claim 11, wherein the at least three ultrasound transducer arrays are selected from the group consisting of: curvilinear array transducer, matrix transducer, linear array transducer, and phased array transducer.

13. The ultrasound transducer of claim 11, wherein the at least three ultrasound transducer arrays comprise curvilinear array transducers.

14. The ultrasound transducer of claim 13, wherein each of the at least three ultrasound transducer arrays includes at least 40 elements.

15. The ultrasound transducer of claim 14, wherein the at least three ultrasound transducer arrays are aligned in an approximately planar configuration.

16. The ultrasound transducer of claim 11, wherein each of the at least three ultrasound transducer arrays has a center frequency of between 2 MHz and 7 MHz.

17. The ultrasound transducer of claim 11,
wherein the at least three transducer arrays are part of a single transducer having independently controllable elements.

18. The ultrasound transducer of claim 11, wherein the central transducer array comprises a curvilinear transducer and wherein the at least two lateral transducer arrays are phased array transducers.

19. A method of measuring fetal blood pressure, comprising the steps of:
providing an ultrasound transducer having at least three ultrasound transducer arrays including a central transducer array and at least two lateral transducer arrays located adjacent the central transducer array, wherein the at least three ultrasound transducer arrays are arranged such that ultrasound beam paths of the at least three ultrasound transducer arrays overlap in an approximately planar location;
obtaining a two-dimensional image of a fetal aorta lumen using the ultrasound transducer;
displaying the two-dimensional image to a user;
obtaining from a user a location of a center of the fetal aorta lumen;
generating a center array echo line from the central transducer array and a plurality of side array echo lines from each of the lateral transducer arrays, wherein each of the center array echo lines and the side array echo lines cross at the location;
obtaining ultrasound data from each of the center array echo line and the plurality of side array echo lines; and
determining fetal blood pressure using the ultrasound data.

20. The method of claim 19, further comprising using electronic beam steering to generate the center array echo line and the plurality of side array echo lines.

21. The method of claim 19, further comprising adjusting a position of the ultrasound transducer such that a center beam of the curved array transducer is approximately perpendicular to a fetal aorta wall.

22. The method of claim 19, further comprising calculating at least one of heart rate, aortic wall thickness, time averaged aortic lumen diameter, pulse wave velocity, local aortic distensibility coefficient, aortic compliance coefficient, elastic modulus of the aortic wall, mean aortic blood flow, stroke volume, downstream peripheral resistance, compliance of the fetal vascular bed, systolic aortic blood pressure, and diastolic aortic blood pressure.

23. The method of claim 19, further comprising tracking at least one of a near wall of the fetal aorta and a far wall of the fetal aorta using the center array echo line.

24. A method of determining a thickness of a fetal aorta wall, comprising the steps of:
obtaining a plurality of ultrasound scans through the fetal aorta wall, wherein each of the plurality of ultrasound scans has a near wall reflection point and a far wall reflection point;
aligning each of the plurality of ultrasound scans according to the near wall reflection point in each of the plurality of ultrasound scans to produce a near wall alignment;
determining a near wall reflection mean from the near wall alignment;
decomposing the near wall reflection mean into a near wall inner Gaussian pulse and a near wall outer Gaussian pulse; and
determining a thickness of the near wall based on the near wall inner Gaussian pulse and the near wall outer Gaussian pulse.

25. The method of claim 24, further comprising:
aligning each of the plurality of ultrasound scans according to the far wall reflection point in each of the plurality of ultrasound scans to produce a far wall alignment;
determining a far wall reflection mean from the far wall alignment;
decomposing the far wall reflection mean into a far wall inner Gaussian pulse and a far wall outer Gaussian pulse; and
determining an inner diameter of the fetal aorta based on the near wall inner Gaussian pulse and the far wall inner Gaussian pulse.

26. A method of displaying multi-angle ultrasound data from a fetus, comprising the steps of:
providing an ultrasound transducer having at least three ultrasound transducer arrays including a central transducer array and at least two lateral transducer arrays located adjacent the central transducer array, wherein the at least three ultrasound transducer arrays are arranged such that ultrasound beam paths of the at least three ultrasound transducer arrays penetrate the fetal tissue and structures from different angles and overlap in an approximately planar location;
obtaining two-dimensional images of the tissue using at least two of the ultrasound transducer arrays; and
combining the two-dimensional images to provide a composite image of the tissue and structures.

27. The method of claim 26, further comprising displaying the composite image to a user, wherein the composite image comprises the anatomical structure related and anisotropic compensated fetal image based on multiple echo amplitudes, tissue angle dependency, and strain properties; aligning the ultrasound transducer with a structure in the tissue;
obtaining from the user a location within the structure;
generating a center array echo line from the central transducer array and a plurality of side array echo lines from each of the lateral transducer arrays, wherein each of the center array echo lines and the side array echo lines cross at the location; and
obtaining ultrasound data from each of the center array echo line and the plurality of side array echo lines.

28. The method of claim 27, wherein the tissue comprises a fetus and wherein the structure comprises an aorta within the fetus, the method further comprising using the ultrasound data to determine at least one of heart rate, aortic wall thickness, time averaged aortic lumen diameter, pulse wave velocity, local aortic distensibility coefficient, aortic compliance coefficient, elastic modulus of the aortic wall, mean aortic blood flow, stroke volume, downstream peripheral resistance, compliance of the fetal vascular bed, systolic aortic blood pressure, diastolic aortic blood pressure, and mean aortic blood pressure.
